SEQUENCE LISTING (4 sequences)

SEQ ID NO: 1 - protein, 121 aa, Camelus sp.
QVQLQESGGGLVQAGDSLRISCRASGLTVNDLYMGWFRQAPGKEREFVGRVTPGDNTDYTYYVDSVKGRFTISRDSAKNTVYLQMNSLKLEDTAVYLCAGRRFGSSEWDYWGQGTQVTVSS

SEQ ID NO: 2 - protein, 10 aa, Camelus sp.
GLTVNDLYMG

SEQ ID NO: 3 - protein, 18 aa, Camelus sp.
RVTPGDNTDYTYYVDSVK

SEQ ID NO: 4 - protein, 10 aa, Camelus sp.
RRFGSSEWDY

(19) United States
(12) Patent Application Publication
(10) Pub. No.: US 2025/0249437 A1
(43) Pub. Date: Aug. 7, 2025

(54) METHODS OF SANITIZING AND/OR REGENERATING A CHROMATOGRAPHY MEDIUM

(71) Applicant: CSL BEHRING AG, Bern (CH)

(72) Inventors: Peter DOLLINGER, Marburg (DE); Dorota SKOTNICKA, Marburg (DE); Roopsee ANAND, Bern (CH)

(21) Appl. No.: 18/855,092

(22) PCT Filed: Apr. 6, 2023
(86) PCT No.: PCT/IB2023/053515

§ 371 Date: Oct. 8, 2024

(30) Foreign Application Priority Data
Apr. 8, 2022 (EP) 22167454.2

(51) Int. Cl. B01J 20/34; A01N 59/00; A01P 1/00; A61L 2/18; A61L 101/02; B01D 15/20; B01D 15/38; B01J 20/26; G01N 33/543

(52) U.S. Cl. CPC B01J 20/3475; A01N 59/00; A01P 1/00; A61L 2/18; B01D 15/203; B01D 15/3809; B01J 20/267; B01J 20/3425; G01N 33/54386; A61L 2101/02

(57) ABSTRACT

The present disclosure relates to methods of sanitizing a chromatography medium. The present disclosure also relates to regenerating the chromatography medium.

### RELATED APPLICATION DATA

The present application claims priority from European Patent Application No. EP22167454.2 filed on 8 Apr. 2022 entitled “Methods of sanitizing and regenerating a chromatography medium”. The entire contents of which is hereby incorporated by reference.

### SEQUENCE LISTING

The present application is filed together with a Sequence Listing in electronic form. The entire contents of the Sequence Listing are hereby incorporated by reference.

### FIELD

The present disclosure relates to methods of sanitizing and/or regenerating a chromatography medium.

### BACKGROUND

The purification of proteins (from natural sources e.g. albumin from plasma and those produced by recombinant techniques) is an important step in the manufacture of proteins, particularly where the protein is for therapeutic use. It is common for several purification steps to be involved in such a manufacturing process to ensure that the protein is of a suitable quality (e.g. purity and stability).

Chromatography-based purification methods are highly used for the purification of proteins. Existing chromatography-based methods of purifying proteins include affinity chromatography (e.g. protein A chromatography), anion exchange chromatography, hydrophobic interaction chromatography, and cation exchange chromatography. An obstacle of existing methods used in the commercial-scale purification of proteins is the high cost given the large volumes of chromatography media required. To address this issue, chromatography media are often re-used. Between cycles of use, a chromatography medium is treated to remove any non-specifically bound proteins that may reduce the ability of the medium to purify protein of interest (i.e., the medium is regenerated) and to sanitize the medium to prevent contamination of future batches of protein of interest (i.e., the medium is sanitized). However, harsh methods used for regenerating and/or sanitizing a chromatography medium may reduce the lifetime of the resin. Several protein based chromatography resins are sensitive to harsh sanitization conditions. For example, aside from newer caustic-resistant ligands, protein A ligands are sensitive to caustic cleaning conditions, such as high sodium hydroxide (NaOH) concentrations, which destroy the ligand over time and reduce its ability to be reused. Thus, there is a need in the art for methods for regenerating and/or sanitizing a chromatography medium that maintains the ability of the medium to purify a protein of interest for multiple cycles of purification.

### SUMMARY

In work leading up to the present invention, the inventors sought to identify a method for sanitizing and/or regenerating a chromatography medium that had been used to purify proteins, e.g., to purify immunoglobulin from serum or plasma. In particular, the inventors recognised that chromatography media comprising protein ligands are sensitive to harsh or caustic sanitisation methods which reduce the resin lifetime. Thus, the inventors sought to identify methods of sanitizing a chromatography medium comprising a ligand (e.g. a protein ligand) that is sensitive to caustic conditions. The inventors tested several options and identified a process for sanitizing a chromatography resin that uses an alkaline detergent. Surprisingly, the inventors found that use of the alkaline detergent, traditionally used as a surface disinfectant, was able to adequately inactivate and clear pathogens from the chromatography medium, without reducing the lifetime of the resin and/or degrading the ligand and/or without significant loss in binding activity of the ligand. The inventors additionally investigated options for regenerating a chromatography medium and found that a combination of ethanol and sodium hydroxide was the most effective for removing residual bound proteins from the medium, e.g., when used in combination with the alkaline detergent for sanitizing the resin. The inventors also found that the use of low concentrations of sodium hydroxide in combination with ethanol for regeneration was particularly effective at removing residual bound proteins from the chromatography medium, and that this combination did not cause precipitation or clogging of the resin, a commonly observed problem with the use of standard high concentrations of sodium hydroxide for regeneration. Furthermore, the inventors found that combining ethanol and sodium hydroxide into a single solution further improved the method by, reducing the volume of solution and/or contact time needed to regenerate the chromatography medium. The methods produced by the inventors allow for multiple cycles of use of the chromatography medium. By increasing the number of cycles that a chromatography medium could effectively be used, the inventors also achieved a significantly lower cost per use of a chromatography medium.

The findings by the inventors provide a method of sanitizing a chromatography medium, the method comprising contacting the medium with an alkaline detergent. In one example, the alkaline detergent comprises an alkaline salt, one or more surfactants and/or a chelator. In one example, the alkaline detergent comprises an alkaline salt, one or more surfactants and, optionally, a chelator. In one example, the alkaline detergent comprises an alkaline salt and one or more surfactants. In one example, the alkaline detergent comprises an alkaline salt, one or more surfactants and a chelator.

In one example, the alkaline detergent is mixed within a solution. In one example, the solution comprises 0.01% to 10%. For example, the solution comprises 0.1% to 10% alkaline detergent. In one example, the solution comprises 0.01% to 5% alkaline detergent. In one example, the solution comprises 0.05% to 5% alkaline detergent. In one example, the solution comprises 0.01% to 5% alkaline detergent. For example, the solution comprises 0.01% to 2.5% alkaline detergent. In another example, the solution comprises 0.01% to 0.1% alkaline detergent. For example, the solution comprises about 0.02% alkaline detergent. In one example, the solution comprises about 0.02% to 0.1% alkaline detergent. In a further example, the solution comprises 0.05% to 0.15% alkaline detergent. In one example, the solution comprises 0.5% to 2.5% alkaline detergent. In one example, the solution comprises 1% to 2.5% alkaline detergent. For example, the solution comprises 2% alkaline detergent.

In one example, the alkaline detergent is CIP-100. For example, the solution comprises 0.1% to 10% CIP-100. In one example, the solution comprises 0.1% to 5% CIP-100. In one example, the solution comprises 0.5% to 5% CIP-100. In one example, the solution comprises 1% to 5% CIP-100. For example, the solution comprises 0.01% to 2.5% CIP-100. In another example, the solution comprises 1% to 2.5% CIP-100. For example, the solution comprises about 2% CIP-100. In a further example, the solution comprises 1.5% to 2.5% CIP-100. In one example, the solution comprises 0.5% to 2.5% CIP-100.

In one example, the alkaline salt is selected from the group consisting of potassium hydroxide, sodium hydroxide, sodium carbonate, sodium acetate, sodium sulphide, and sodium bicarbonate. For example, the alkaline salt is potassium hydroxide or sodium hydroxide. In one example, the alkaline salt is potassium hydroxide. In another example, the alkaline salt is sodium hydroxide.

In one example, the alkaline detergent comprises between 10 mM and 150 mM alkaline salt. For example, the alkaline detergent comprises between 20 mM and 150 mM alkaline salt. In another example, the alkaline detergent comprises between 30 mM and 140 mM alkaline salt. In a further example, the alkaline detergent comprises between 40 mM and 130 mM alkaline salt. For example, the alkaline detergent comprises between 50 mM and 120 mM alkaline salt. In one example, the alkaline detergent comprises between 50 mM and 100 mM alkaline salt. In another example, the alkaline detergent comprises between 60 mM and 90 mM alkaline salt. In a further example, the alkaline detergent comprises between 60 mM and 80 mM alkaline salt. For example, the alkaline detergent comprises between 70 mM and 80 mM alkaline salt. In one example, the alkaline detergent comprises about 75 mM alkaline salt.

In one example, the alkaline detergent comprises 10 mM, or 15 mM, or 20 mM, or 25 mM, or 30 mM, or 35 mM, or 40 mM, or 45 mM, or 50 mM, or 55 mM, or 60 mM, or 65 mM, or 70 mM, or 75 mM, or 80 mM, or 85 mM, or 90 mM, 95 mM, or 100 mM, or 105 mM, or 110 mM, or 115 mM, or 120 mM, or 125 mM, or 130 mM, or 135 mM, or 140 mM, or 145 mM, or 150 mM of alkaline salt.

In one example, the alkaline salt is potassium hydroxide or sodium hydroxide, wherein the alkaline salt is at a concentration of between 20 mM and 150 mM. For example, the alkaline salt is potassium hydroxide or sodium hydroxide, wherein the alkaline salt is at a concentration of between 50 mM and 100 mM. In one example, the alkaline salt is potassium hydroxide or sodium hydroxide, wherein the alkaline salt is at a concentration of between 60 mM and 80 mM. For example, the alkaline salt is potassium hydroxide or sodium hydroxide, wherein the alkaline salt is at a concentration of about 75 mM.

In one example, the alkaline salt is potassium hydroxide at a concentration of between 20 mM and 150 mM. For example, the alkaline salt is potassium hydroxide at a concentration of between 50 mM and 100 mM. In one example, the alkaline salt is potassium hydroxide at a concentration of between 60 mM and 80 mM. For example, the alkaline salt is potassium hydroxide at a concentration of about 75 mM.

In one example, the alkaline salt is sodium hydroxide at a concentration of 20 mM to 150 mM. For example, the alkaline salt is sodium hydroxide at a concentration of between 50 mM and 100 mM. In one example, the alkaline salt is sodium hydroxide at a concentration of between 60 mM and 80 mM. For example, the alkaline salt is sodium hydroxide at a concentration of about 75 mM.

In one example, the one or more surfactants is selected from the group consisting of amine ethoxylates, amphocarboxylates, triethanolamine, Octyl-β-Dglucopyranoside (OGP), Polysorbate, Poloxamer, Lauryldimethyl-amine (LDAO), Myristyldimethylamine-N-oxide (TDAO), Sodium-cholate, Decyl-ß-D-glucopyranoside and Dodecyl maltoside. For example, the Polysorbate is Polysorbate 80 or Polysorbate 20.

In one example, the surfactant is a Polysorbate, such as Polysorbate 20.

In one example, the surfactant is TDAO. Commercially available TDAO solutions will be apparent to the skilled person and include, for example, Deviron® C16. In one example, the surfactant is Deviron® C16.

In one example, the surfactant is TDAO at a concentration of 0.1% to 10% (w/v). In one example, the surfactant is TDAO at a concentration of 0.1% to 5% (w/v). For example, the surfactant is TDAO at a concentration of 0.01% to 2% (w/v). In another example, the surfactant is TDAO at a concentration of 0.01% to 0.1% (w/v). In one example, the surfactant is TDAO at a concentration of 0.02% to 0.1% (w/v). For example, the surfactant is TDAO at a concentration of 0.02% (w/v). In a further example, the surfactant is TDAO at a concentration of 0.05% to 0.15% (w/v). In one example, the surfactant is TDAO at a concentration of 0.5% to 2% (w/v).

In one example, the alkaline detergent comprises TDAO and an alkaline salt selected from potassium hydroxide and sodium hydroxide.

In one example, the alkaline detergent comprises TDAO and potassium hydroxide. In one example, the alkaline detergent comprises between 10 mM to 150 mM of potassium hydroxide and between 0.01% to 2% (w/v) of TDAO. In one example, the alkaline detergent comprises between 60 mM to 80 mM of potassium hydroxide and between 0.01% to 0.1% (w/v) of TDAO. In one example, the alkaline detergent comprises between 60 mM to 80 mM of potassium hydroxide and between 0.02% to 0.1% (w/v) of TDAO. In one example, the alkaline detergent comprises 75 mM of potassium hydroxide and between 0.02% to 0.1% (w/v) of TDAO. For example, the alkaline detergent comprises about 0.02% TDAO and about 75 mM of potassium hydroxide.

In one example, the alkaline detergent comprises TDAO and sodium hydroxide. In one example, the alkaline detergent comprises between 10 mM to 150 mM of sodium hydroxide and between 0.01% to 2% (w/v) of TDAO. In one example, the alkaline detergent comprises between 60 mM to 80 mM of sodium hydroxide and between 0.01% to 0.1% (w/v) of TDAO. In one example, the alkaline detergent comprises between 60 mM to 80 mM of sodium hydroxide and between 0.02% to 0.1% (w/v) of TDAO. In one example, the alkaline detergent comprises 75 mM of sodium hydroxide and between 0.02% to 0.1% (w/v) of TDAO. For example, the alkaline detergent comprises about 0.02% TDAO and about 75 mM of sodium hydroxide.

In one example, the surfactant and the alkaline salt are mixed in combination. For example, the surfactant and the alkaline salt are in the same solution (i.e., mixed in combination in the same solution). In another example, the surfactant and the alkaline salt are in separate solutions. It will be apparent to the skilled person from the disclosure herein that when the surfactant and the alkaline salt are in separate solutions, that the medium is sanitized by contacting the medium with the surfactant and the alkaline salt simultaneous or sequentially. For example, the medium is sanitized by contacting the medium with the alkaline salt followed by the surfactant. In another example, the medium is sanitized by contacting the medium with the surfactant followed by the alkaline salt. In an exemplary method of the disclosure, the medium is sanitized by contacting the medium with 0.02% to 0.1% surfactant followed by 75 mM of the alkaline salt selected from sodium hydroxide and potassium hydroxide.

In one example, the chelator is ethylenediaminetetraacetic acid (EDTA), ethylene glycol-bis(β-aminoethyl ether)-N,N,N′,N′-tetraacetic acid (EGTA), ethylenediamine-N,N′-disuccinic acid (EDDS), iminodisuccinic acid (IDS), methylglycinediacetic acid (MGDA), triethylenetetramine (Trien), iminodiacetic acid (IDA), nitrilotriacetic acid (NTA), tripolyphosphate (TPP), diethylenetriaminepentaacetic acid (DTPA), sodium diethyldithiocarbamate (DDC), L-Glutamic acid N,N-diacetic acid, tetrasodium salt (GLDA), and penicillamine, sodium gluconate, trisodium citrate, DMSA, DMPS, BAPTA, ALA and a salt thereof

In one example, the chelator is a biodegradable chelator, such as ethylenediamine-N,N′-disuccinic acid (EDDS), iminodisuccinic acid (IDS), methylglycinediacetic acid (MGDA) or nitrilotriacetic acid (NTA).

In some examples, a chromatography medium is contacted with multiple column volumes (CVs) of the alkaline detergent to sanitize the medium. In this regard, a volume of alkaline detergent approximately equal to the volume of the chromatography medium flowed through the medium is equal to one CV. In some examples, 1 or 2 or 3 or 4 or 5 or 6 or 7 or 8 or 9 or 10 column volumes of a solution are flowed through the chromatography medium.

In some examples, a chromatography medium is contacted with an alkaline detergent to sanitize the medium for a defined period of time. For example, the period of time is 10 minutes or 15 minutes or 20 minutes or 30 minutes or 45 minutes or 60 minutes. In one example, the period of time is 10 minutes or 15 minutes or 20 minutes or 30 minutes or 45 minutes or 60 minutes or 75 minutes or 90 minutes. In one example, the period of time is 10 minutes. In one example, the period of time is 15 minutes. In one example, the period of time is 20 minutes. In one example, the period of time is 30 minutes. In one example, the period of time is 45 minutes. In one example, the period of time is 60 minutes. In one example, the period of time is 75 minutes. In one example, the period of time is 90 minutes.

In one example, the method comprises contacting the medium with an alkaline detergent for a defined period of time. For example, the period of time is 10 minutes or 15 minutes or 20 minutes or 30 minutes or 45 minutes or 60 minutes. In one example, the method comprises contacting the medium with an alkaline detergent for 10 minutes or 15 minutes or 20 minutes or 30 minutes or 45 minutes or 60 minutes or 75 minutes or 90 minutes. In one example, the period of time is 10 minutes. In one example, the method comprises contacting the medium with an alkaline detergent for 15 minutes. In one example, the method comprises contacting the medium with an alkaline detergent for 20 minutes. In one example, the method comprises contacting the medium with an alkaline detergent for 30 minutes. In one example, the method comprises contacting the medium with an alkaline detergent for 45 minutes. In one example, the method comprises contacting the medium with an alkaline detergent for 60 minutes. In one example, the method comprises contacting the medium with an alkaline detergent for 75 minutes. In one example, the method comprises contacting the medium with an alkaline detergent for 90 minutes.

In some forms of the present disclosure a method as described herein additionally comprises regenerating the chromatography medium. For example, the method comprises regenerating the chromatography medium prior to sanitizing the chromatography medium.

Methods for regenerating a chromatography medium are known in the art and will be apparent to the skilled person. For example, regenerating a chromatography medium includes contacting a chromatography medium with a solution including but not limited to, salt solutions (e.g., NaCl), caustic solutions (e.g., up to 1.5 M sodium hydroxide), solvents such as for example hydrochloric acid, acetic acid, ethanol, methanol, isopropanol and acetonitrile, Tris or Tris EDTA or compounds such as urea or guanidine hydrochloride. In one example, regenerating a chromatography medium includes contacting a chromatography medium with a polar solution.

Some regeneration methods include contacting the medium with a solution comprising ethylene glycol, sodium chloride, sodium hydroxide or a combination of sodium hydroxide and ethanol.

In some examples, the medium is regenerated by contacting the medium with a solution comprising ethanol, e.g., 5-30% ethanol, such as 20% ethanol. In some examples, the medium is regenerated by including one or more steps of washing the resin with one or more column volumes of ethanol; followed by washing the resin with one or more column volumes of ultrapure water.

In some examples, regenerating a chromatography medium comprises contacting the medium with a combination of solutions. In some examples, the medium is regenerated by including one or more steps of washing the resin with one or more column volumes of 1 M NaCl and 1 M sodium hydroxide; washing the resin with one or more column volumes of ultrapure water; washing the resin with one or more column volumes of 1M acetic acid; followed by washing the resin with one or more column volumes of ultrapure water. Additional methods and reagents for regenerating a chromatography resin are described herein.

In some examples, the medium is regenerated by using a combination of NaCl and sodium hydroxide, for example by washing with a combination of one or more column volumes of 2.0 M NaCl and one or more column volumes of NaOH.

In some examples, the medium is regenerated by contacting the medium with ethanol, e.g., 5-30% ethanol, such as 20% ethanol followed by sodium hydroxide, e.g., 5-30 mM sodium hydroxide, e.g., 20 mM sodium hydroxide.

In some examples, the ethanol and the sodium hydroxide are applied sequentially. For example, the medium is regenerated by contacting the medium with ethanol followed by sodium hydroxide. In another example, the medium is regenerated by contacting the medium with sodium hydroxide followed by ethanol. In some examples, the ethanol and the sodium hydroxide are applied simultaneously as separate solutions. In one example, the ethanol and the sodium hydroxide are applied as a single solution.

In some examples, the medium is regenerated by contacting the resin with 0.2 M NaOH. In one examples, the medium is regenerated by contacting the resin with 5 mM NaOH.

In some examples, a chromatography medium is contacted with multiple column volumes (CVs) of a solution to regenerate the column. In this regard, a volume of solution approximately equal to the volume of the chromatography medium flowed through the medium is equal to one CV. In some examples, 1 or 2 or 3 or 4 or 5 or 6 or 7 or 8 or 9 or 10 column volumes of a solution are flowed through the chromatography medium.

In some examples, a chromatography medium is contacted with a solution to regenerate the column for a defined period of time. For example, the period of time is 10 minutes or 15 minutes or 20 minutes or 30 minutes or 45 minutes or 60 minutes.

The present disclosure additionally provides a method of sanitizing and regenerating a chromatography medium. For example, the method comprises sanitizing the chromatography medium followed by regenerating the chromatography medium. In another example, the method comprises regenerating the chromatography medium followed by sanitizing the chromatography medium.

The present disclosure provides a method of sanitizing and regenerating a chromatography medium, the method comprising:
(i) sanitizing the chromatography medium by contacting the medium with an alkaline detergent, and
(ii) regenerating the chromatography medium by contacting the medium with a regeneration solution comprising ethylene glycol, sodium chloride, sodium hydroxide or a combination of sodium hydroxide and ethanol.

In one example, the method comprises:
(i) sanitizing the chromatography medium by contacting the medium with an alkaline detergent comprising an alkaline salt, one or more surfactants and, optionally, a chelator; and
(ii) regenerating the chromatography medium by contacting the medium with a regeneration solution comprising a combination of sodium hydroxide and ethanol.

In one example, the method comprises:
(i) sanitizing the chromatography medium by contacting the medium with an alkaline detergent comprising between 0.01% to 10% (w/v) of one or more surfactants and/or between 10 mM and 150 mM of an alkaline salt; and
(ii) regenerating the chromatography medium by contacting the medium with a regeneration solution comprising a combination of 5-30 mM sodium hydroxide and 5-30% ethanol.

In one example, the method comprises:
(i) sanitizing the chromatography medium by contacting the medium with an alkaline detergent comprising between 0.01% to 10% (w/v) of one or more surfactants and/or between 10 mM and 150 mM of an alkaline salt selected from sodium hydroxide and potassium hydroxide; and
(ii) regenerating the chromatography medium by contacting the medium with a regeneration solution comprising a combination of 5-30 mM sodium hydroxide and 5-30% ethanol.

In one example, the method comprises:
(i) sanitizing the chromatography medium by contacting the medium with an alkaline detergent comprising between 0.01% to 5% (w/v) of one or more surfactants and/or between 50 mM and 100 mM of an alkaline salt selected from sodium hydroxide and potassium hydroxide; and
(ii) regenerating the chromatography medium by contacting the medium with a regeneration solution comprising a combination of 5-30 mM sodium hydroxide and 5-30% ethanol.

In one example, the method comprises:
(i) sanitizing the chromatography medium by contacting the medium with an alkaline detergent comprising between 0.01% to 1% (w/v) of one or more surfactants and/or between 60 mM and 80 mM of an alkaline salt selected from sodium hydroxide and potassium hydroxide; and
(ii) regenerating the chromatography medium by contacting the medium with a regeneration solution comprising a combination of 5-30 mM sodium hydroxide and 5-30% ethanol.

In one example, the method comprises:
(i) sanitizing the chromatography medium by contacting the medium with an alkaline detergent comprising between 0.02% to 0.1% (w/v) of one or more surfactants and/or between 60 mM and 80 mM of an alkaline salt selected from sodium hydroxide and potassium hydroxide; and
(ii) regenerating the chromatography medium by contacting the medium with a regeneration solution comprising a combination of 5-30 mM sodium hydroxide and 5-30% ethanol.

In one example, the method comprises:
(i) sanitizing the chromatography medium by contacting the medium with an alkaline detergent comprising 0.02% (w/v) of one or more surfactants and/or 75 mM of an alkaline salt selected from sodium hydroxide and potassium hydroxide; and
(ii) regenerating the chromatography medium by contacting the medium with a regeneration solution comprising a combination of 5 mM sodium hydroxide and 20% ethanol.

In one example, the method comprises:
(i) sanitizing the chromatography medium by contacting the medium with an alkaline detergent comprising 0.02% (w/v) of one or more surfactants and/or 75 mM of sodium hydroxide; and
(ii) regenerating the chromatography medium by contacting the medium with a regeneration solution comprising a combination of 5 mM sodium hydroxide and 20% ethanol.

In one example, the method comprises:
(i) sanitizing the chromatography medium by contacting the medium with an alkaline detergent comprising 0.02% (w/v) of one or more surfactants and/or 75 mM of potassium hydroxide; and
(ii) regenerating the chromatography medium by contacting the medium with a regeneration solution comprising a combination of 5 mM sodium hydroxide and 20% ethanol.

In one example, the method comprises:
(i) sanitizing the chromatography medium by contacting the medium with an alkaline detergent comprising 0.02% (w/v) of one or more surfactants and/or 75 mM of sodium hydroxide; and
(ii) regenerating the chromatography medium by contacting the medium with a regeneration solution comprising a combination of 20 mM sodium hydroxide and 20% ethanol.

In one example, the method comprises:
(i) sanitizing the chromatography medium by contacting the medium with an alkaline detergent comprising 0.02% (w/v) of one or more surfactants and/or 75 mM of potassium hydroxide; and
(ii) regenerating the chromatography medium by contacting the medium with a regeneration solution comprising a combination of 20 mM sodium hydroxide and 20% ethanol.

In one example, the method comprises:
(i) sanitizing the chromatography medium by contacting the medium with an alkaline detergent comprising CIP-100; and
(ii) regenerating the chromatography medium by contacting the medium with a regeneration solution comprising a combination of sodium hydroxide and ethanol.

In one example, the method comprises:
(i) sanitizing the chromatography medium by contacting the medium with an alkaline detergent comprising 1%-5% CIP-100; and
(ii) regenerating the chromatography medium by contacting the medium with a regeneration solution comprising a combination of 5-30 mM sodium hydroxide and 5-30% ethanol.

In one example, the method comprises:
(i) sanitizing the chromatography medium by contacting the medium with an alkaline detergent comprising 2% CIP-100; and
(ii) regenerating the chromatography medium by contacting the medium with a regeneration solution comprising a combination of 5 mM or 20 mM sodium hydroxide and 20% ethanol.

In one example, the method comprises:
(i) sanitizing the chromatography medium by contacting the medium with an alkaline detergent comprising 2% CIP-100; and
(ii) regenerating the chromatography medium by contacting the medium with a regeneration solution comprising a combination of 5 mM sodium hydroxide and 20% ethanol.

In one example, the method comprises:
(i) sanitizing the chromatography medium by contacting the medium with an alkaline detergent comprising 2% CIP-100; and
(ii) regenerating the chromatography medium by contacting the medium with a regeneration solution comprising a combination of 20 mM sodium hydroxide and 20% ethanol.

In one example, the method comprises:
(i) sanitizing the chromatography medium by contacting the medium with an alkaline detergent comprising Myristyldimethylamine-N-oxide (TDAO) and an alkaline salt selected from potassium hydroxide and sodium hydroxide; and
(ii) regenerating the chromatography medium by contacting the medium with a regeneration solution comprising a combination of sodium hydroxide and ethanol.

In one example, the method comprises:
(i) sanitizing the chromatography medium by contacting the medium with an alkaline detergent comprising between 0.01% to 10% (w/v) of Myristyldimethylamine-N-oxide (TDAO) and between 10 mM and 150 mM of an alkaline salt selected from potassium hydroxide and sodium hydroxide; and
(ii) regenerating the chromatography medium by contacting the medium with a regeneration solution comprising a combination of 5-30 mM sodium hydroxide and 5-30% ethanol.

In one example, the method comprises:
(i) sanitizing the chromatography medium by contacting the medium with an alkaline detergent comprising between 0.01% to 1% (w/v) of Myristyldimethylamine-N-oxide (TDAO) and between 60 mM and 80 mM of an alkaline salt selected from potassium hydroxide and sodium hydroxide; and
(ii) regenerating the chromatography medium by contacting the medium with a regeneration solution comprising a combination of 5 or 20 mM sodium hydroxide and 20% ethanol.

In one example, the method comprises:
(i) sanitizing the chromatography medium by contacting the medium with an alkaline detergent comprising between 0.02% to 0.1% (w/v) of Myristyldimethylamine-N-oxide (TDAO) and between 60 mM and 80 mM of an alkaline salt selected from potassium hydroxide and sodium hydroxide; and
(ii) regenerating the chromatography medium by contacting the medium with a regeneration solution comprising a combination of 5 or 20 mM sodium hydroxide and 20% ethanol.

In one example, the method comprises:
(i) sanitizing the chromatography medium by contacting the medium with an alkaline detergent comprising between 0.01% to 1% (w/v) of Myristyldimethylamine-N-oxide (TDAO) and between 60 mM and 80 mM of potassium hydroxide; and
(ii) regenerating the chromatography medium by contacting the medium with a regeneration solution comprising a combination of 5 or 20 mM sodium hydroxide and 20% ethanol.

In one example, the method comprises:
(i) sanitizing the chromatography medium by contacting the medium with an alkaline detergent comprising between 0.02% to 0.1% (w/v) of Myristyldimethylamine-N-oxide (TDAO) and between 60 mM and 80 mM of potassium hydroxide; and
(ii) regenerating the chromatography medium by contacting the medium with a regeneration solution comprising a combination of 5 or 20 mM sodium hydroxide and 20% ethanol.

In one example, the method comprises:
(i) sanitizing the chromatography medium by contacting the medium with an alkaline detergent comprising between 0.01% to 1% (w/v) of Myristyldimethylamine-N-oxide (TDAO) and between 60 mM and 80 mM of sodium hydroxide; and
(ii) regenerating the chromatography medium by contacting the medium with a regeneration solution comprising a combination of 5 or 20 mM sodium hydroxide and 20% ethanol.

In one example, the method comprises:
(i) sanitizing the chromatography medium by contacting the medium with an alkaline detergent comprising between 0.02% to 0.1% (w/v) of Myristyldimethylamine-N-oxide (TDAO) and between 60 mM and 80 mM of sodium hydroxide; and
(ii) regenerating the chromatography medium by contacting the medium with a regeneration solution comprising a combination of 5 or 20 mM sodium hydroxide and 20% ethanol.

In one example, the method comprises:
(i) sanitizing the chromatography medium by contacting the medium with an alkaline detergent comprising 0.02% (w/v) of Myristyldimethylamine-N-oxide (TDAO) and 75 mM of an alkaline salt selected from potassium hydroxide and sodium hydroxide; and
(ii) regenerating the chromatography medium by contacting the medium with a regeneration solution comprising a combination of 20 mM sodium hydroxide and 20% ethanol.

In one example, the method comprises:
(i) sanitizing the chromatography medium by contacting the medium with an alkaline detergent comprising 0.02% (w/v) of Myristyldimethylamine-N-oxide (TDAO) and 75 mM of potassium hydroxide; and
(ii) regenerating the chromatography medium by contacting the medium with a regeneration solution comprising a combination of 20 mM sodium hydroxide and 20% ethanol.

In one example, the method comprises:
(i) sanitizing the chromatography medium by contacting the medium with an alkaline detergent comprising 0.02% (w/v) of Myristyldimethylamine-N-oxide (TDAO) and 75 mM of sodium hydroxide; and
(ii) regenerating the chromatography medium by contacting the medium with a regeneration solution comprising a combination of 20 mM sodium hydroxide and 20% ethanol.

In one example, the method comprises:
(i) sanitizing the chromatography medium by contacting the medium with an alkaline detergent comprising 0.02% (w/v) of Myristyldimethylamine-N-oxide (TDAO) and 75 mM of potassium hydroxide; and
(ii) regenerating the chromatography medium by contacting the medium with a regeneration solution comprising a combination of 5 mM sodium hydroxide and 20% ethanol.

In one example, the method comprises:
(i) sanitizing the chromatography medium by contacting the medium with an alkaline detergent comprising 0.02% (w/v) of Myristyldimethylamine-N-oxide (TDAO) and 75 mM of sodium hydroxide; and
(ii) regenerating the chromatography medium by contacting the medium with a regeneration solution comprising a combination of 5 mM sodium hydroxide and 20% ethanol.

The present disclosure provides a method of regenerating and sanitizing a chromatography medium, the method comprising:
(i) regenerating the chromatography medium by contacting the medium with a regeneration solution comprising ethylene glycol, sodium chloride, sodium hydroxide or a combination of sodium hydroxide and ethanol; and
(ii) sanitizing the chromatography medium by contacting the medium with an alkaline detergent.

In one example, the method comprises:
(i) regenerating the chromatography medium by contacting the medium with a regeneration solution comprising a combination of sodium hydroxide and ethanol and
(ii) sanitizing the chromatography medium by contacting the medium with an alkaline detergent comprising an alkaline salt, one or more surfactants and, optionally, a chelator.

In one example, the method comprises:
(i) regenerating the chromatography medium by contacting the medium with a regeneration solution comprising a combination of 5-30 mM sodium hydroxide and 5-30% ethanol; and
(ii) sanitizing the chromatography medium by contacting the medium with an alkaline detergent comprising between 0.01% to 10% (w/v) of one or more surfactants and/or between 10 mM and 150 mM of an alkaline salt.

In one example, the method comprises:
(i) regenerating the chromatography medium by contacting the medium with a regeneration solution comprising a combination of 5-30 mM sodium hydroxide and 5-30% ethanol; and
(ii) sanitizing the chromatography medium by contacting the medium with an alkaline detergent comprising between 0.01% to 10% (w/v) of one or more surfactants and/or between 10 mM and 150 mM of an alkaline salt selected from sodium hydroxide and potassium hydroxide.

In one example, the method comprises:
(i) regenerating the chromatography medium by contacting the medium with a regeneration solution comprising a combination of 5-30 mM sodium hydroxide and 5-30% ethanol; and
(ii) sanitizing the chromatography medium by contacting the medium with an alkaline detergent comprising between 0.01% to 5% (w/v) of one or more surfactants and/or between 50 mM and 100 mM of an alkaline salt selected from sodium hydroxide and potassium hydroxide.

In one example, the method comprises:
(i) regenerating the chromatography medium by contacting the medium with a regeneration solution comprising a combination of 5-30 mM sodium hydroxide and 5-30% ethanol; and
(ii) sanitizing the chromatography medium by contacting the medium with an alkaline detergent comprising between 0.01% to 1% (w/v) of one or more surfactants and/or between 60 mM and 80 mM of an alkaline salt selected from sodium hydroxide and potassium hydroxide.

In one example, the method comprises:
(i) regenerating the chromatography medium by contacting the medium with a regeneration solution comprising a combination of 5-30 mM sodium hydroxide and 5-30% ethanol; and
(ii) sanitizing the chromatography medium by contacting the medium with an alkaline detergent comprising between 0.02% to 0.1% (w/v) of one or more surfactants and/or between 60 mM and 80 mM of an alkaline salt selected from sodium hydroxide and potassium hydroxide.

In one example, the method comprises:
(i) regenerating the chromatography medium by contacting the medium with a regeneration solution comprising a combination of 5 mM sodium hydroxide and 20% ethanol; and
(ii) sanitizing the chromatography medium by contacting the medium with an alkaline detergent comprising 0.02% (w/v) of one or more surfactants and/or 75 mM of an alkaline salt selected from sodium hydroxide and potassium hydroxide.

In one example, the method comprises:
(i) regenerating the chromatography medium by contacting the medium with a regeneration solution comprising a combination of 5 mM sodium hydroxide and 20% ethanol; and
(ii) sanitizing the chromatography medium by contacting the medium with an alkaline detergent comprising 0.02% (w/v) of one or more surfactants and/or 75 mM of sodium hydroxide.

In one example, the method comprises:
(i) regenerating the chromatography medium by contacting the medium with a regeneration solution comprising a combination of 5 mM sodium hydroxide and 20% ethanol; and
(ii) sanitizing the chromatography medium by contacting the medium with an alkaline detergent comprising 0.02% (w/v) of one or more surfactants and/or 75 mM of potassium hydroxide.

In one example, the method comprises:
(i) regenerating the chromatography medium by contacting the medium with a regeneration solution comprising a combination of 20 mM sodium hydroxide and 20% ethanol; and
(ii) sanitizing the chromatography medium by contacting the medium with an alkaline detergent comprising 0.02% (w/v) of one or more surfactants and/or 75 mM of sodium hydroxide.

In one example, the method comprises:
(i) regenerating the chromatography medium by contacting the medium with a regeneration solution comprising a combination of 20 mM sodium hydroxide and 20% ethanol; and
(ii) sanitizing the chromatography medium by contacting the medium with an alkaline detergent comprising 0.02% (w/v) of one or more surfactants and/or 75 mM of potassium hydroxide.

In one example, the method comprises:
(i) regenerating the chromatography medium by contacting the medium with a regeneration solution comprising a combination of sodium hydroxide and ethanol; and
(ii) sanitizing the chromatography medium by contacting the medium with an alkaline detergent comprising CIP-100.

In one example, the method comprises:
(i) regenerating the chromatography medium by contacting the medium with a regeneration solution comprising a combination of 5-30 mM sodium hydroxide and 5-30% ethanol; and
(ii) sanitizing the chromatography medium by contacting the medium with an alkaline detergent comprising 1%-5% CIP-100.

In one example, the method comprises:
(i) regenerating the chromatography medium by contacting the medium with a regeneration solution comprising a combination of 5 mM or 20 mM sodium hydroxide and 20% ethanol; and
(ii) sanitizing the chromatography medium by contacting the medium with an alkaline detergent comprising 2% CIP-100.

In one example, the method comprises:
(i) regenerating the chromatography medium by contacting the medium with a regeneration solution comprising a combination of 5 mM sodium hydroxide and 20% ethanol; and
(i) sanitizing the chromatography medium by contacting the medium with an alkaline detergent comprising 2% CIP-100.

In one example, the method comprises:
(i) regenerating the chromatography medium by contacting the medium with a regeneration solution comprising a combination of 20 mM sodium hydroxide and 20% ethanol; and
(ii) sanitizing the chromatography medium by contacting the medium with an alkaline detergent comprising 2% CIP-100.

In one example, the method comprises:
(i) regenerating the chromatography medium by contacting the medium with a regeneration solution comprising a combination of sodium hydroxide and ethanol; and
(ii) sanitizing the chromatography medium by contacting the medium with an alkaline detergent comprising Myristyldimethylamine-N-oxide (TDAO) and an alkaline salt selected from potassium hydroxide and sodium hydroxide.

In one example, the method comprises:
(i) regenerating the chromatography medium by contacting the medium with a regeneration solution comprising a combination of 5-30 mM sodium hydroxide and 5-30% ethanol; and
(ii) sanitizing the chromatography medium by contacting the medium with an alkaline detergent comprising between 0.01% to 10% (w/v) of Myristyldimethylamine-N-oxide (TDAO) and between 10 mM and 150 mM of an alkaline salt selected from potassium hydroxide and sodium hydroxide.

In one example, the method comprises:
(i) regenerating the chromatography medium by contacting the medium with a regeneration solution comprising a combination of 5 or 20 mM sodium hydroxide and 20% ethanol; and
(ii) sanitizing the chromatography medium by contacting the medium with an alkaline detergent comprising between 0.01% to 1% (w/v) of Myristyldimethylamine-N-oxide (TDAO) and between 60 mM and 80 mM of an alkaline salt selected from potassium hydroxide and sodium hydroxide.

In one example, the method comprises:
(i) regenerating the chromatography medium by contacting the medium with a regeneration solution comprising a combination of 5 or 20 mM sodium hydroxide and 20% ethanol; and
(ii) sanitizing the chromatography medium by contacting the medium with an alkaline detergent comprising between 0.02% to 0.1% (w/v) of Myristyldimethylamine-N-oxide (TDAO) and between 60 mM and 80 mM of an alkaline salt selected from potassium hydroxide and sodium hydroxide.

In one example, the method comprises:
(i) regenerating the chromatography medium by contacting the medium with a regeneration solution comprising a combination of 5 or 20 mM sodium hydroxide and 20% ethanol; and
(ii) sanitizing the chromatography medium by contacting the medium with an alkaline detergent comprising between 0.01% to 1% (w/v) of Myristyldimethylamine-N-oxide (TDAO) and between 60 mM and 80 mM of potassium hydroxide.

In one example, the method comprises:
(i) regenerating the chromatography medium by contacting the medium with a regeneration solution comprising a combination of 5 or 20 mM sodium hydroxide and 20% ethanol; and
(ii) sanitizing the chromatography medium by contacting the medium with an alkaline detergent comprising between 0.02% to 0.1% (w/v) of Myristyldimethylamine-N-oxide (TDAO) and between 60 mM and 80 mM of potassium hydroxide.

In one example, the method comprises:
(i) regenerating the chromatography medium by contacting the medium with a regeneration solution comprising a combination of 5 or 20 mM sodium hydroxide and 20% ethanol; and
(ii) sanitizing the chromatography medium by contacting the medium with an alkaline detergent comprising between 0.01% to 1% (w/v) of Myristyldimethylamine-N-oxide (TDAO) and between 60 mM and 80 mM of sodium hydroxide.

In one example, the method comprises:
(i) regenerating the chromatography medium by contacting the medium with a regeneration solution comprising a combination of 5 or 20 mM sodium hydroxide and 20% ethanol; and
(ii) sanitizing the chromatography medium by contacting the medium with an alkaline detergent comprising between 0.02% to 0.1% (w/v) of Myristyldimethylamine-N-oxide (TDAO) and between 60 mM and 80 mM of sodium hydroxide.

In one example, the method comprises:
(i) regenerating the chromatography medium by contacting the medium with a regeneration solution comprising a combination of 20 mM sodium hydroxide and 20% ethanol; and
(ii) sanitizing the chromatography medium by contacting the medium with an alkaline detergent comprising 0.02% (w/v) of Myristyldimethylamine-N-oxide (TDAO) and 75 mM of an alkaline salt selected from potassium hydroxide and sodium hydroxide.

In one example, the method comprises:
(i) regenerating the chromatography medium by contacting the medium with a regeneration solution comprising a combination of 20 mM sodium hydroxide and 20% ethanol; and
(ii) sanitizing the chromatography medium by contacting the medium with an alkaline detergent comprising 0.02% (w/v) of Myristyldimethylamine-N-oxide (TDAO) and 75 mM of potassium hydroxide.

In one example, the method comprises:
(i) regenerating the chromatography medium by contacting the medium with a regeneration solution comprising a combination of 20 mM sodium hydroxide and 20% ethanol; and
(ii) sanitizing the chromatography medium by contacting the medium with an alkaline detergent comprising 0.02% (w/v) of Myristyldimethylamine-N-oxide (TDAO) and 75 mM of sodium hydroxide.

In one example, the method comprises:
(i) regenerating the chromatography medium by contacting the medium with a regeneration solution comprising a combination of 5 mM sodium hydroxide and 20% ethanol; and
(ii) sanitizing the chromatography medium by contacting the medium with an alkaline detergent comprising 0.02% (w/v) of Myristyldimethylamine-N-oxide (TDAO) and 75 mM of potassium hydroxide.

In one example, the method comprises:
(i) regenerating the chromatography medium by contacting the medium with a regeneration solution comprising a combination of 5 mM sodium hydroxide and 20% ethanol; and
(ii) sanitizing the chromatography medium by contacting the medium with an alkaline detergent comprising 0.02% (w/v) of Myristyldimethylamine-N-oxide (TDAO) and 75 mM of sodium hydroxide.

In some examples, the chromatography medium is an ion exchange medium, an affinity medium, a hydrophobic interaction chromatography medium, a size exclusion column or a mixed mode chromatography medium. For example, the medium is an affinity chromatography medium, e.g., an affinity chromatography medium that binds to an Fc region of an antibody. Exemplary affinity chromatography media comprises protein A or an antibody binding fragment thereof, protein G or an antibody binding fragment thereof or a ligand capable of specifically binding to a CH1 or CH3 domain of human IgG. In one example, the ligand capable of specifically binding to a CH3 domain of human IgG comprises a camelid-derived single domain [VHH] antibody fragment.

In some examples, the medium is not caustic stable or alkaline stable.

In some examples, the medium is an affinity chromatography medium that is not caustic stable or alkaline stable.

In one example, the chromatography medium comprises a matrix selected from the group consisting of poly(styrene-divinylbenzene), cross-linked poly(styrene-divinylbenzene), silica, agarose, cross-linked agarose, controlled pore glass, polymethacrylate and cellulose. In an exemplified form of the disclosure the chromatography medium comprises cross-linked poly(styrene-divinylbenzene).

In one example, the chromatography medium comprises a VHH antibody fragment capable of specifically binding to a CH3 domain of human IgG conjugated to a cross-linked poly(styrene-divinylbenzene) matrix.

The present disclosure provides a method comprising contacting a chromatography medium comprising a VHH antibody fragment capable of specifically binding to a CH3 domain of human IgG conjugated to a cross-linked poly(styrene-divinylbenzene) matrix with an alkaline detergent comprising an alkaline salt, one or more surfactants and/or a chelator.

In one example, the alkaline detergent comprises an alkaline salt, one or more surfactants and, optionally, a chelator. In one example, the alkaline detergent comprises an alkaline salt and one or more surfactants. In one example, the alkaline detergent comprises an alkaline salt, one or more surfactants and a chelator.

The present disclosure additionally provides a method comprising regenerating chromatography medium comprising a VHH antibody fragment capable of specifically binding to a CH3 domain of human IgG conjugated to a cross-linked poly(styrene-divinylbenzene) matrix by contacting the medium with a solution comprising a combination sodium hydroxide and ethanol and sanitizing the medium by contacting the medium with an alkaline detergent comprising an alkaline salt, one or more surfactants and/or a chelator.

In one example, the alkaline detergent comprises an alkaline salt, one or more surfactants and, optionally, a chelator. In one example, the alkaline detergent comprises an alkaline salt and one or more surfactants. In one example, the alkaline detergent comprises an alkaline salt, one or more surfactants and a chelator.

In one example, the chromatography medium has been previously contacted with a sample. For example, the sample is plasma or a fraction thereof.

In one example, the plasma is clarified cryo-rich plasma.

In one example, the plasma fraction is clarified cryo-poor plasma.

In some examples, a method described herein is performed as part of a process of continuous chromatography.

In one example, the method described herein is repeated on the resin wherein multiple batches of plasma or fractions thereof are applied to the resin. For example, the method described herein is repeated on the resin after each batch of plasma or fraction thereof.

It will be apparent to the skilled person from the disclosure herein that the number of cycles per batch of plasma or fraction thereof will be dependent on the volume and/or weight of plasma or fraction thereof.

In one example, the method described herein is performed on the resin after each batch of plasma or fractions thereof, wherein a batch comprises at least 1 cycle. In another example, a batch comprises at least 5 cycles. For example, a batch comprises at least 10 cycles. In a further example, a batch comprises at least 15 cycles. In another example, a batch comprises at least 20 cycles. In one example, a batch comprises at least 25 cycles. In one example, a batch comprises at least 30 cycles. In another example, a batch comprises at least 35 cycles. In a further example, a batch comprises at least 40 cycles. In one example, a batch comprises at least 45 cycles. In another example, a batch comprises at least 50 cycles. In a further example, a batch comprises at least 60 cycles, or at least 70 cycles, or at least 80 cycles, or at least 90 cycles, or at least 100 cycles. In one example, a batch comprises between 1 and 100 cycles. For example, a batch comprises between 10 and 100 cycles. In another example, a batch comprises between 20 and 80 cycles. In a further example, a batch comprises between 30 and 60 cycles.

In one example, a batch comprises 1 cycle. In another example, a batch comprises at least 5 cycles. For example, a batch comprises 10 cycles. In a further example, a batch comprises 15 cycles. In another example, a batch comprises 20 cycles. In one example, a batch comprises 25 cycles. In one example, a batch comprises 30 cycles. In another example, a batch comprises 35 cycles. In a further example, a batch comprises 40 cycles. In one example, a batch comprises 45 cycles. In another example, a batch comprises 50 cycles. In a further example, a batch comprises 60 cycles, or 70 cycles, or 80 cycles, or 90 cycles, or 100 cycles.

In one example, the method described herein is repeated on the resin after at least 50 cycles of plasma or fraction thereof. For example, the method is repeated on the resin after at least 50 cycles per batch of plasma or fraction thereof. In an example, the method is repeated on the resin after between 50 to 80 cycles, 60 to 80 cycles, or 70 to 80 cycles per batch of plasma or fraction thereof. For example, the method is repeated on the resin after at least 60, or 65, or 70, or 75, or 80, cycles per batch of plasma or fraction thereof.

In one example, the method is repeated on the resin after each batch of plasma or fractions thereof, wherein multiple batches of plasma or fractions thereof are applied to the resin. In one example, the method is repeated on the resin after each batch of plasma or fraction thereof, wherein 2, or 3, or 4, or 5, or 6, or 7, or 8, or 9, or 10 batches of plasma or fractions thereof are applied to the resin. In one example, the method is repeated on the resin after each batch of plasma or fraction thereof, wherein between 4 to 10 batches of plasma or fractions thereof are applied to the resin.

In one example, the regeneration and/or sanitisation method described herein is performed on the resin after each individual cycle. In another example, the regeneration and/or sanitisation method described herein is performed on the resin after multiple cycles. For example, the regeneration and/or sanitisation method described herein is performed on the resin after at least 50 cycles. In one example, the regeneration and/or sanitisation method described herein is performed on the resin after at least 100 cycles. In another example, the regeneration and/or sanitisation method described herein is performed on the resin after at least 150 cycles. In a further example, the regeneration and/or sanitisation method described herein is performed on the resin after at least 200 cycles. In one example, the regeneration and/or sanitisation method described herein performed on the resin after each batch of plasma or fractions thereof. For example, the regeneration and/or sanitisation method described herein is performed on the resin between each batch of plasma or fractions thereof, i.e., before the loading of each batch of plasma or fractions thereof on to the resin.

In one example, the regeneration step is performed on the resin as part of each individual cycle. For example, the regeneration step is performed on the resin after each individual cycle.

In one example, the sanitisation step is performed after each batch of plasma or fractions thereof. For example, the sanitisation step is performed after multiple cycles of plasma or fractions thereof. In one example, the sanitisation step is performed after each batch of plasma or fractions thereof, wherein each batch comprises at least 50 cycles.

In one example, the regeneration step is performed on the resin after each individual cycle and the sanitisation step is performed after each batch of plasma or fractions thereof.

It will be apparent to the skilled person from the disclosure herein that performing the method of the disclosure on the resin between each batch of plasma or fraction thereof extends the lifetime of the resin. For example, the method of the disclosure extends the lifetime of the resin or enables reuse of the resin for up to a total of 800 cycles. In one example, the method of the disclosure extends the lifetime of the resin or enables reuse of the resin for up to a total of 100, or 200, or 300, or 400, or 500, or 600, or 700 cycles. For example, the method of the disclosure extends the lifetime of the resin or enables reuse of the resin for up to a total of 100 cycles. For example, the method of the disclosure extends the lifetime of the resin or enables reuse of the resin for up to a total of 200 cycles. For example, the method of the disclosure extends the lifetime of the resin or enables reuse of the resin for up to a total of 300 cycles. For example, the method of the disclosure extends the lifetime of the resin or enables reuse of the resin for up to a total of 400 cycles. For example, the method of the disclosure extends the lifetime of the resin or enables reuse of the resin for up to a total of 500 cycles. For example, the method of the disclosure extends the lifetime of the resin or enables reuse of the resin for up to a total of 600 cycles. For example, the method of the disclosure extends the lifetime of the resin or enables reuse of the resin for up to a total of 700 cycles.

In one example, the resin is reused up to a total of up to 500 cycles with up to 10 batches of plasma or fractions thereof, wherein the method of the disclosure is performed on the resin between each batch of plasma or fraction thereof.

In one example, the continuous chromatography is continuous affinity chromatography.

In one example, the continuous affinity chromatography is selected from the group consisting of simulated moving bed (SMB) chromatography, periodic counter-current chromatography (PCC), continuous counter-current tangential chromatography (CCTC), and continuous counter-current spiral chromatography (CCSC).

In one example, the continuous affinity chromatography is simulated moving bed (SMB) chromatography. In another example, the continuous affinity chromatography is periodic counter-current chromatography (PCC). In a further example, the continuous affinity chromatography is continuous counter-current tangential chromatography (CCTC). In one example, the continuous affinity chromatography is continuous counter-current spiral chromatography (CCSC).

In one example, the continuous affinity chromatography process is performed at a pressure in the range of about 2 to about 5 bar. For example, the continuous affinity chromatography process is performed at a pressure in the range of about 2 to about 4 bar. In one example, the continuous affinity chromatography process is performed at a pressure in the range of about 2 to about 3.5 bar.

In one example, a method as performed herein is for purifying IgG from plasma or a fraction thereof and yields greater than 85% or 90% or 95% of total IgG loaded onto the chromatography medium, wherein the chromatography medium has been exposed to the method of sanitization as described herein at least one time or five times or ten times.

In one example, a method as performed herein is for purifying IgG from plasma or a fraction thereof and yields greater than 85% or 90% or 95% of total IgG loaded onto the chromatography medium, wherein the chromatography medium has been exposed to the method of regeneration and sanitization as described herein at least one time or five times or ten times.

In one example, the method is performed at large scale. For example, the method is performed on an industrial or a commercial scale. Methods of performing on an industrial or a commercial scale will be apparent to a skilled person and/or described herein. For example, the method performed on an industrial scale comprises large scale purification of IgG from the plasma or fraction thereof.

In one example, large scale purification of IgG is performed using at least 500 kg of the plasma or fraction thereof. For example, large scale purification of IgG is performed using between 500 kg to 1000 kg, or 1000 kg to 2500 kg, or 2500 kg to 5000 kg, or 5000 kg to 7500 kg, or 7500 kg, or 10000 kg, or 10000 kg to 12500 kg, or 12500 kg to 15000 kg of the plasma or fraction thereof. In one example, large scale purification of IgG is performed using at least 1000 kg, or 2500 kg, or 5000 kg, or 7500 kg, or 10000 kg, or 12500 kg, or 15000 kg of the plasma of fraction thereof. In one example, large scale purification of IgG is performed using at least 1000 kg of the plasma of fraction thereof.

In one example, large scale purification of IgG is performed using at least 2500 kg of the plasma of fraction thereof. In one example, large scale purification of IgG is performed using at least 5000 kg of the plasma of fraction thereof. In one example, large scale purification of IgG is performed using at least 7500 kg of the plasma of fraction thereof. In one example, large scale purification of IgG is performed using at least 10000 kg of the plasma of fraction thereof. In one example, large scale purification of IgG is performed using at least 12500 kg of the plasma of fraction thereof. In one example, large scale purification of IgG is performed using at least 15000 kg of the plasma of fraction thereof.

In one example, a method as performed herein is for purifying IgG from plasma or a fraction thereof and yields a solution comprising less than 9 mg albumin per g IgG and/or less than 5 mg IgM per g IgG and/or less than 7 mg IgA per g IgG.

In one example, a method as performed herein is for purifying IgG from plasma or a fraction thereof and yields IgG having a purity greater than 95%.

In one example, a method of sanitization as described herein reduces the level of virus or prion in a chromatography medium. In one example, a method of sanitization as described herein reduces the level of virus in a chromatography medium.

In one example, the level of the virus or prion is reduced at least 1 log or 2 logs or 3 logs or 4 logs or 5 logs. For example, the level of the virus or prion is reduced at least 3 logs. For example, the level of the virus or prion is reduced at least 4 logs. For example, the level of the virus or prion is reduced to below the level of quantification of the assay used to detect the virus or prion.

In one example, a method of the disclosure permits cleaning in place, i.e., can be or is performed on a chromatography medium without removing the medium from, e.g., a column or the column in which the medium is contained from its position within a facility.

### BRIEF DESCRIPTION OF THE DRAWINGS

FIG. 1 is a graphical representation of a chromatogram showing results of regenerating a FcXP chromatography medium using 20% ethanol followed by 20 mM sodium hydroxide and then sanitizing the medium using 2% CIP-100.

FIG. 2 includes two graphical representations showing binding capacity of two FcXP chromatography media (columns 1 and 2) following purification of eight batches of IgG from plasma, regeneration with 20% ethanol followed by 20 mM sodium hydroxide and sanitization using 2% CIP-100.

FIG. 3 is a graphical representation showing impurity levels (albumin, IgM and IgA as indicated) per gram of IgG in eluate from a FcXP chromatography medium following purification of ten batches of IgG from plasma, regeneration with 20% ethanol followed by 20 mM sodium hydroxide and sanitization using 2% CIP-100. Data for batches 1, 5 and 10 are shown.

FIG. 4 is a graphical representation showing % purity of IgG in eluate from a FcXP chromatography medium following purification of ten batches of IgG from plasma, regeneration with 20% ethanol followed by 20 mM sodium hydroxide and sanitization using 2% CIP-100. Data for batches 1, 5 and 10 are shown.

FIGS. 5A and 5B are graphical representations showing inactivation of BVDV following treatment with 2% CIP-100 at room temperature (RT). Samples are as indicated. Viral titre are shown.

FIG. 6 is a graphical representation showing inactivation of hepatitis A virus following treatment with 2% CIP-100 at room temperature (RT). Samples are as indicated. Viral titre are shown.

FIG. 7 is a graphical representation showing the feed pump pressure profile from a continuous chromatography lifetime study of 10 batches (1 batch=50 product cycles) with regeneration (ethanol and sodium hydroxide) and sanitization performed between each batch.

### KEY TO SEQUENCE LISTING

SEQ ID NO: 1 is an amino acid sequence of VHH fragment
SEQ ID NO: 2 is an amino acid sequence of CDR1 of the VHH fragment
SEQ ID NO: 3 is an amino acid sequence of CDR2 of the VHH fragment
SEQ ID NO: 4 is an amino acid sequence of CDR3 of the VHH fragment

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

Furthermore, as used herein the singular forms of “a”, “and” and “the” include plural references unless the context clearly dictates otherwise.

### Selected Definitions

The term “sanitization” in the context of a chromatography medium shall be taken to mean a process, which substantially inactivates and/or removes microorganisms (including pathogenic and other bacteria, vegetative forms, and spores) and/or prions on and/or in the chromatography medium. In one example, sanitization of the chromatography medium reduces microorganisms on and/or in the chromatography medium by 5%, 10%, 15%, 25%, 30%, 35%, 40%, 50%, 60%, 70%, 80%, 90%, or 100% compared to a chromatography medium not sanitized by the method of the present disclosure.

In one example, sanitization of the chromatography medium inactivates microorganisms on and/or in the chromatography medium by 5%, 10%, 15%, 25%, 30%, 35%, 40%, 50%, 60%, 70%, 80%, 90%, or 100% compared to a chromatography medium not sanitized by the method of the present disclosure. In one example, sanitization includes sterilization. Exemplary microorganisms which may be sanitized from the chromatography medium by the method of the present disclosure include fungi, bacteria, viruses, protista, mildew, molds, and mold spores. For example, the microorganisms includes viral (parvovirus B19, human immunodeficiency virus (HIV), hepatitis viruses, human herpes viruses, cytomegalovirus, Epstein-Barr virus, West Nile virus), or bacteria (Treponema pallidum, Neisseria gonorrhoea, Chlamydia trachomatis, Streptococcus pyogenes, Mycobacterium tuberculosis, Brucella melitensis, Brucella melitensis, Ehrlichia, Staphylococci, Pseudomonas aeruginos) or parasitic (Plasmodium, Trypanosoma cruzi, Babesia microti). As discussed above, “sanitization” also includes inactivating and/or removing prions.

The term “regeneration” in the context of a chromatography medium shall be taken to mean a process where the strength and/or properties of the chromatography medium is partially or substantially restored to allow the use of the chromatography medium in one or more further chromatography cycles. Regeneration can increase the effectiveness of the chromatography media while extending its useful life. In one example, regeneration of the chromatography medium involves the removal of adsorbed proteins from the chromatography medium.

The term “chromatography medium” shall be taken to mean a solid or semi solid phase for use in chromatography. In one example, a chromatography medium is made up of a porous or non-porous support to which a plurality of ligands are attached, examples of which are described herein. For example, the chromatography medium is a chromatography resin. Exemplary chromatography resins include MabSelect® SuRe® (Cytiva), MabSelect® SuRe® LX (Cytiva), POROS® PI50 (ThermoFischer), CaptureSelect® FcXP affinity chromatography resins (Thermo Fisher), CaptureSelect® FcXL affinity resin (Thermo Fisher), CaptureSelect® IgG-CH1 affinity resin (Thermo Fisher), and CaptureSelect® FcXP agarose affinity resin (Thermo Fisher). Further exemplary affinity chromatography resins include IgSelect® affinity resin (Cytiva), HiTrap® IgSelect® affinity resin (Cytiva), Pierce® Protein G agarose affinity resin (Thermo Fisher), and Protein G sepharose 4 fast flow affinity resin (Cytiva). For example, the chromatography medium is a chromatography membrane. Exemplary chromatography membranes include Purexa® A (Purilogics), Mustang Q (Pall Corporation).

The term “ligand” shall be taken to mean a molecule immobilised to a matrix of the chromatography medium, for example, to an affinity chromatography resin which specifically binds to the CH3 domain of human IgG. For example, the ligand is a camelid-derived single domain [VHH] antibody fragment.

The term “matrix” shall be taken to mean a support to which the ligand is immobilised. Exemplary matrices are cross-linked poly(styrene-divinylbenzene) matrix and agarose-based matrix.

The term “alkaline detergent” shall be taken to mean a water-soluble agent with an ability to dissolve fats, oils and grease. Alkaline detergents typically comprise alkaline salts (e.g. potassium hydroxide and sodium hydroxide), one or more surfactants and/or a chelator. Exemplary alkaline detergents include CIP-100 (Steris Life Sciences), ProKlenz One (Steris Life Sciences), ProKlenz-100 (Steris Life Sciences) and COSA CIP-92 (Ecolab). In one example, the alkaline detergent comprises 2% CIP-100.

The term “contacting” shall include, but not limited to methods of washing, immersing, dipping, bathing, soaking, storing, spraying or flushing the chromatography medium with an alkaline detergent.

The term “alkaline salt” shall be taken to mean a basic, ionic salt of an alkali metal or alkaline earth metal element. Exemplary alkaline salts include potassium hydroxide or sodium hydroxide.

The term “surfactants” shall be taken to refer to a substance which tends to reduce the surface tension of a liquid in which it is dissolved. Surfactants may act as a dispersant, foaming agent, emulsifier, wetting agent or detergent.

The term “chelator” shall be taken to refer to a compound that form stable, water-soluble complexes with metal ions.

The term “ion exchange medium” shall be taken to mean a chromatography medium comprising a negatively charged or positively charged functional group. For example, an ion exchange medium includes anion exchange chromatography medium and cation exchange chromatography medium. Anion exchange chromatography medium is a positively charged medium with an affinity for molecules (e.g. proteins) having net negative surface charges. Exemplary anion exchange chromatography medium include POROS® PI50 anion resin (ThermoFisher), POROS® XQ anion resin (ThermoFisher), POROS® HQ 50 anion resin (ThermoFisher), and POROS® D50 anion resin (ThermoFisher). Cation exchange chromatography medium is a negatively charged medium with an affinity for molecules (e.g. proteins) having net positive surface charges. Exemplary cation exchange chromatography medium include POROS® XS strong cation exchange resin (ThermoFisher) and POROS® HS strong cation exchange resin (ThermoFisher).

The term “affinity chromatography medium” shall be taken to mean a chromatography medium comprising an affinity chromatography ligand (e.g. camelid-derived single domain [VHH] antibody fragment, or protein A or an antibody fragment thereof, or protein G or an antibody fragment thereof) attached to a matrix such as, e.g., those described herein. Exemplary affinity chromatography medium used in a method described herein include POROS® CaptureSelect® FcXP affinity resin (Thermo Fisher) and CaptureSelect® FcXP agarose affinity resin (Thermo Fisher). Further exemplary affinity chromatography medium include a medium having an amino acid sequence encoded by SEQ ID NO: 1 or variants thereof that specifically bind to the CH3 domain of human IgG. In one example, an exemplary affinity chromatography medium comprises a VHH antigen-binding protein comprising a complementarity determining region (CDR) 1, CDR2 and/or CDR3 having an amino acid sequence of SEQ ID NO: 2, 3 or 4 respectively. Exemplary affinity chromatography resins are also described in U.S. Pat. No. 10,259,886. Exemplary protein A chromatography medium include MabSelect® PrismA protein A resins (Cytiva), Praesto® Jetted A50 protein A resins (Purolite Corp.), and Amsphere® A3 (JSR Corp.), MabSelect® SuRe® protein A resins (Cytiva), and MabSelect® SuRe® LX protein A resins (Cytiva). Exemplary protein G chromatography medium include Protein G Sepharose 4 Fast Flow resin (Cytiva), Protein G Resin (abbexa), Dynabeads® Protein G Magnetic Beads (ThermoFisher), Pierce® Protein G Agarose (ThermoFisher), Pierce® Protein G Plus Agarose (ThermoFisher), POROS® MabCapture® G Select (ThermoFisher), PROTEINDEX® rProtein G Agarose (Margvelgent Biosciences), and ProteinIso® Protein G Resin (TransGen Biotech Co., LTD).

The term “hydrophobic interaction chromatography medium” shall be taken to mean a chromatography medium comprising a hydrophobic ligand attached to a matrix such as, e.g., those described herein. Hydrophobic interaction chromatography (HIC) comprising a hydrophobic interaction chromatography medium separates molecules (e.g. proteins) based on the molecules hydrophobicity (i.e. aversion to water). Exemplary hydrophobic interaction chromatography medium include POROS® Ethyl Hydrophobic Interaction Chromatography (HIC) resin (ThermoFisher) and POROS® Benzyl Ultra Hydrophobic Interaction Chromatography (HIC) resin (ThermoFisher).

The term “mixed mode chromatography medium” shall be taken to mean a chromatography medium which allows for the separation of molecules (e.g. proteins) based on more than one form of interaction between the molecules and the medium. For example, mixed mode chromatography medium may comprise affinity chromatography ligands and ion exchange functional groups attached to a matrix. Exemplary mixed mode chromatography medium include CMM HyperCel (Satorius), MEP HyperCel (Satorius), HEA HyperCel (Sartorius), PPA HyperCel (Sartorius), and HA Ultragel® (Satorius).

The term “specifically binds”, “specifically binding” or “binds specifically” shall be taken to mean that a protein of the disclosure reacts or associates more frequently, more rapidly, with greater duration and/or with greater affinity with a particular antigen or cell expressing same than it does with alternative antigens or cells. For example, a ligand capable of specifically binding to a CH3 domain of human IgG with materially greater affinity (e.g., 1.5 fold or 2 fold or 5 fold or 10 fold or 20 fold or 40 fold or 60 fold or 80 fold to 100 fold or 150 fold or 200 fold) than it does to other antigens. Generally, but not necessarily, reference to binding means specific binding, and each term shall be understood to provide explicit support for the other term.

The term “immunoglobulin G (IgG)”, also known as “gamma globulin” or “immune globulin”, shall be taken to mean antibody of isotype G. There are several subclasses of IgG, for example, IgG1, IgG2, IgG3 and IgG4.

The term “camelid-derived single domain [VHH] antibody fragment” shall be taken to mean a VHH domain of a camelidae antibody. The camelidae antibody is an antibody produced from camels and llamas and has no CH1 domain normally present in human immunoglobulins and only one VHH domain. Exemplary affinity chromatography resins comprising the camelid-derived [VHH] antibody fragment include CaptureSelect® antibody affinity chromatography resins (Thermo Fisher). For example, CaptureSelect® FcXL affinity resin, POROS® CaptureSelect® FcXP affinity resin, CaptureSelect IgG-CH1 affinity resin, and CaptureSelect FcXP agarose affinity resin.

The term “dynamic binding capacity” or “DBC” of a chromatography medium shall be taken to refer to the maximum amount of protein that the medium will bind under operating conditions before significant breakthrough of unbound protein occurs.

The term “plasma” shall refer to the straw-coloured/pale yellow component of blood obtained from one or more blood donor(s). Methods of obtaining plasma from a donor will be apparent to a skilled person and/or described herein. For example, plasma is obtained by removing red blood cells from donated blood. For example, plasma is obtained by plasmapheresis.

The term “plasma fraction” or “fraction thereof” shall refer to plasma which has been fractionated to isolate one or more desirable protein components from the plasma. For example, plasma may be fractionated to isolate cryo-precipitates (proteins that precipitate out of solution when a unit of fresh frozen plasma is slowly thawed in the cold) and cryo-supernatant (also known as cryo-poor plasma). For example, plasma may be fractionated by ethanol precipitation to produce IgG-containing Oncley fractions, Cohn fractions, ammonium sulphate precipitates, or Precipitate A (KN A) from plasma as described in U.S. Pat. No. 3,301,842. Plasma fractions include II+III precipitate produced according to Cohn methods such as Method 6, Cohn et. al. J. Am; Chem. Soc., 68 (3), 459-475 (1946), Method 9, Oncley et al. J. Am; Chem. Soc., 71, 541-550 (1946), or the I+II+III precipitate, Method 10, Cohn et. al. J. Am; Chem. Soc., 72, 465-474 (1950); as well as the method of Deutsch et. al. J. Biol. Chem. 164, 109-118 (1946) or the Precipitate-A of Nitschmann and Kistler Vox Sang. 7, 414-424 (1962); Helv. Chim. Acta 37, 866-873 (1954). For example, the plasma may be fractionated by octanoic acid fractionation as described in European application 893450. Typically, Cohn Fractions, Kistler/Nitschmann Precipitate A (KN A) exist as a suspended paste. Other purification techniques including chromatography may be used.

The term “cryo-precipitate” or “cryo-precipitates” refers to proteins in plasma that precipitate out of solution when a unit of fresh frozen plasma is slowly thawed in the cold. Cryo-precipitates include factor VIII, fibrinogen, von Willebrand factor, factor XIII and platelet membrane microparticles.

The term “cryo-poor plasma” shall be taken to mean plasma removed of cryo-precipitates.

The term “cryo-rich plasma” shall be taken to mean plasma comprising components typically found in cryo-precipitates.

### Sanitization

The present disclosure provides a method of sanitizing a chromatography medium. Suitable chromatography medium for use in the method will be apparent to the skilled person and/or described herein. In one example, the chromatography medium is used in ion exchange chromatography (e.g. anion or cation exchange chromatography), affinity chromatography (e.g. protein A or protein G chromatography), mixed-mode chromatography, or size exclusion chromatography. For example, after a chromatography cycle wherein the chromatography medium is used in one or more round of equilibration, sample loading, binding, washing and/or elution, the chromatography medium is sanitized. Sanitization of the chromatography medium inactivates and/or removes microorganisms from the medium. In one example, sanitization of the chromatography medium inactivates microorganisms on the medium. In one example, sanitization of the chromatography medium removes microorganisms from the medium.

For example, sanitization of the chromatography medium reduces microorganisms on and/or in the chromatography medium by 5% compared to a chromatography medium not sanitized by the method of the present disclosure. In one example, sanitization of the chromatography medium reduces microorganisms on and/or in the chromatography medium by 10% compared to a chromatography medium not sanitized by the method of the present disclosure. In one example, sanitization of the chromatography medium reduces microorganisms on and/or in the chromatography medium by 15% compared to a chromatography medium not sanitized by the method of the present disclosure. In one example, sanitization of the chromatography medium reduces microorganisms on and/or in the chromatography medium by 20% compared to a chromatography medium not sanitized by the method of the present disclosure. In one example, sanitization of the chromatography medium reduces microorganisms on and/or in the chromatography medium by 25% compared to a chromatography medium not sanitized by the method of the present disclosure. In one example, sanitization of the chromatography medium reduces microorganisms on and/or in the chromatography medium by 30% compared to a chromatography medium not sanitized by the method of the present disclosure. In one example, sanitization of the chromatography medium reduces microorganisms on and/or in the chromatography medium by 35% compared to a chromatography medium not sanitized by the method of the present disclosure. In one example, sanitization of the chromatography medium reduces microorganisms on and/or in the chromatography medium by 40% compared to a chromatography medium not sanitized by the method of the present disclosure. In one example, sanitization of the chromatography medium reduces microorganisms on and/or in the chromatography medium by 50% compared to a chromatography medium not sanitized by the method of the present disclosure. In one example, sanitization of the chromatography medium reduces microorganisms on and/or in the chromatography medium by 60% compared to a chromatography medium not sanitized by the method of the present disclosure. In one example, sanitization of the chromatography medium reduces microorganisms on and/or in the chromatography medium by 70% compared to a chromatography medium not sanitized by the method of the present disclosure. In one example, sanitization of the chromatography medium reduces microorganisms on and/or in the chromatography medium by 80% compared to a chromatography medium not sanitized by the method of the present disclosure. In one example, sanitization of the chromatography medium reduces microorganisms on and/or in the chromatography medium by 90% compared to a chromatography medium not sanitized by the method of the present disclosure. In one example, sanitization of the chromatography medium reduces microorganisms on and/or in the chromatography medium by 100% compared to a chromatography medium not sanitized by the method of the present disclosure.

In another example, sanitization of the chromatography medium inactivates microorganisms on and/or in the chromatography medium by 5% compared to a chromatography medium not sanitized by the method of the present disclosure. In one example, sanitization of the chromatography medium inactivates microorganisms on and/or in the chromatography medium by 10% compared to a chromatography medium not sanitized by the method of the present disclosure. In one example, sanitization of the chromatography medium inactivates microorganisms on and/or in the chromatography medium by 15% compared to a chromatography medium not sanitized by the method of the present disclosure. In one example, sanitization of the chromatography medium inactivates microorganisms on and/or in the chromatography medium by 20% compared to a chromatography medium not sanitized by the method of the present disclosure. In one example, sanitization of the chromatography medium inactivates microorganisms on and/or in the chromatography medium by 25% compared to a chromatography medium not sanitized by the method of the present disclosure. In one example, sanitization of the chromatography medium inactivates microorganisms on and/or in the chromatography medium by 30% compared to a chromatography medium not sanitized by the method of the present disclosure. In one example, sanitization of the chromatography medium inactivates microorganisms on and/or in the chromatography medium by 35% compared to a chromatography medium not sanitized by the method of the present disclosure. In one example, sanitization of the chromatography medium inactivates microorganisms on and/or in the chromatography medium by 40% compared to a chromatography medium not sanitized by the method of the present disclosure. In one example, sanitization of the chromatography medium inactivates microorganisms on and/or in the chromatography medium by 50% compared to a chromatography medium not sanitized by the method of the present disclosure. In one example, sanitization of the chromatography medium inactivates microorganisms on and/or in the chromatography medium by 60% compared to a chromatography medium not sanitized by the method of the present disclosure. In one example, sanitization of the chromatography medium inactivates microorganisms on and/or in the chromatography medium by 70% compared to a chromatography medium not sanitized by the method of the present disclosure. In one example, sanitization of the chromatography medium inactivates microorganisms on and/or in the chromatography medium by 80% compared to a chromatography medium not sanitized by the method of the present disclosure. In one example, sanitization of the chromatography medium inactivates microorganisms on and/or in the chromatography medium by 90% compared to a chromatography medium not sanitized by the method of the present disclosure. In one example, sanitization of the chromatography medium inactivates microorganisms on and/or in the chromatography medium by 100% compared to a chromatography medium not sanitized by the method of the present disclosure.

Suitable alkaline detergents for use in the method of the present disclosure will be apparent to the skilled person and/or described herein. Typically, alkaline detergents comprise an alkaline salt, one or more surfactants and/or a chelator. Exemplary commercially available alkaline detergents include CIP-100 (Steris Life Sciences), ProKlenz One (Steris Life Sciences) ProKlenz-100 (Steris Life Sciences) and COSA CIP-92 (Ecolab). For example, the method of sanitization may comprises contacting the chromatography medium with 2% CIP-100 (Steris Life Sciences).

In one example, the alkaline detergent is mixed within a solution. For example, the solution comprises 0.1% to 10% alkaline detergent. For example, the solution comprises 0.2% to 9% or 0.3% to 8% or 0.4% to 7% alkaline detergent. In one example, the solution comprises 0.5% to 5% alkaline detergent. In one example, the solution comprises 1% or 2% or 3% or 4% or 5% alkaline detergent. In one example, the solution comprises 1% alkaline detergent. For example, the solution comprises 2% alkaline detergent. In one example, the solution comprises 3% alkaline detergent. In one example, the solution comprises 4% alkaline detergent. In one example, the solution comprises 5% alkaline detergent.

In one example, the alkaline detergent comprises an alkaline salt, one or more surfactants and/or a chelator. In one example, the alkaline detergent comprises an alkaline salt and one or more surfactants. In one example, the alkaline detergent comprises an alkaline salt, one or more surfactants and a chelator

In one example, the alkaline salt is selected from the group consisting of potassium hydroxide, sodium hydroxide, sodium carbonate, sodium acetate, sodium sulphide, and sodium bicarbonate. For example, the alkaline salt is potassium hydroxide. For example, the alkaline salt is sodium hydroxide. For example, the alkaline salt is sodium carbonate. For example, the alkaline salt is sodium acetate. For example, the alkaline salt is sodium sulphide. For example, the alkaline salt is sodium bicarbonate.

In one example, the alkaline salt is potassium hydroxide or sodium hydroxide.

In one example, the alkaline detergent comprises between 5% to 45%, or 5% to 10%, or 10% to 15%, or 15% to 20%, or 20% to 25%, or 25% to 30%, or 30% to 35%, or 35% to 40%, or 40% to 45% (w/v) of alkaline salt. For example, the alkaline detergent comprises between 5% to 10% (w/v) of alkaline salt. For example, the alkaline detergent comprises between 10% to 15% (w/v) of alkaline salt. For example, the alkaline detergent comprises between 15% to 20% (w/v) of alkaline salt. For example, the alkaline detergent comprises between 20% to 25% (w/v) of alkaline salt. For example, the alkaline detergent comprises between 25% to 30% (w/v) of alkaline salt. For example, the alkaline detergent comprises between 30% to 35% (w/v) of alkaline salt. For example, the alkaline detergent comprises between 35% to 40% (w/v) of alkaline salt. For example, the alkaline detergent comprises between 40% to 45% (w/v) of alkaline salt.

In one example, the alkaline detergent comprises at least 5%, 10%, 15%, 20%, 25%, 30%, 35%, 40%, or 45% (w/v) of alkaline salt. For example, the alkaline detergent comprises at least 5% (w/v) of alkaline salt. For example, the alkaline detergent comprises at least 10% (w/v) of alkaline salt. For example, the alkaline detergent comprises at least 15% (w/v) of alkaline salt. For example, the alkaline detergent comprises at least 20% (w/v) of alkaline salt. For example, the alkaline detergent comprises at least 25% (w/v) of alkaline salt. For example, the alkaline detergent comprises at least 30% (w/v) of alkaline salt. For example, the alkaline detergent comprises at least 35% (w/v) of alkaline salt. For example, the alkaline detergent comprises at least 40% (w/v) of alkaline salt. For example, the alkaline detergent comprises at least 45% (w/v) of alkaline salt.

In one example, the alkaline detergent comprises 5%, 10%, 15%, 20%, 25%, 30%, 35%, 40%, or 45% (w/v) of alkaline salt. For example, the alkaline detergent comprises 5% (w/v) of alkaline salt. For example, the alkaline detergent comprises 10% (w/v) of alkaline salt. For example, the alkaline detergent comprises 15% (w/v) of alkaline salt. For example, the alkaline detergent comprises 20% (w/v) of alkaline salt. For example, the alkaline detergent comprises 25% (w/v) of alkaline salt. For example, the alkaline detergent comprises 30% (w/v) of alkaline salt. For example, the alkaline detergent comprises 35% (w/v) of alkaline salt. For example, the alkaline detergent comprises 40% (w/v) of alkaline salt. For example, the alkaline detergent comprises 45% (w/v) of alkaline salt.

In one example, the alkaline detergent comprises between 10 mM to 15 mM, or 15 mM to 20 mM, or 20 mM to 30 mM, or 30 mM to 40 mM, or 40 mM to 50 mM, or 50 mM to 60 mM, or 60 mM to 70 mM, or 70 mM to 80 mM, or 80 mM to 90 mM of alkaline salt.

In one example, the alkaline detergent comprises between 10 mM to 15 mM, or 15 mM to 20 mM, or 20 mM to 30 mM, or 30 mM to 40 mM, or 40 mM to 50 mM, or 50 mM to 60 mM, or 60 mM to 70 mM, or 70 mM to 80 mM, or 80 mM to 90 mM, or 90 mM to 100 mM, or 100 mM to 110 mM, or 110 mM to 120 mM, or 120 mM to 130 mM, or 130 mM to 140 mM, or 140 mM to 150 mM of alkaline salt. For example, the alkaline detergent comprises between 10 mM to 15 mM of alkaline salt. For example, the alkaline detergent comprises between 15 mM to 20 mM of alkaline salt. For example, the alkaline detergent comprises between 20 mM to 30 mM of alkaline salt. For example, the alkaline detergent comprises between 30 mM to 40 mM of alkaline salt. For example, the alkaline detergent comprises between 40 mM to 50 mM of alkaline salt. For example, the alkaline detergent comprises between 50 mM to 60 mM of alkaline salt. For example, the alkaline detergent comprises between 60 mM to 70 mM of alkaline salt. For example, the alkaline detergent comprises between 70 mM to 80 mM of alkaline salt. For example, the alkaline detergent comprises between 80 mM to 90 mM of alkaline salt. For example, the alkaline detergent comprises between 90 mM to 100 mM of alkaline salt. For example, the alkaline detergent comprises between 100 mM to 110 mM of alkaline salt. For example, the alkaline detergent comprises between 110 mM to 120 mM of alkaline salt. For example, the alkaline detergent comprises between 120 mM to 130 mM of alkaline salt. For example, the alkaline detergent comprises between 130 mM to 140 mM of alkaline salt. For example, the alkaline detergent comprises between 140 mM to 150 mM of alkaline salt.

In one example, the alkaline detergent comprises at least 10 mM, 15 mM, 20 mM, 30 mM, 40 mM, 50 mM, 60 mM, 70 mM, 80 mM, or 90 mM of alkaline salt. In one example, the alkaline detergent comprises at least 10 mM, 15 mM, 20 mM, 30 mM, 40 mM, 50 mM, 60 mM, 70 mM, 80 mM, or 90 mM, or 100 mM, or 110 mM, or 120 mM, or 130 mM, or 140 mM of alkaline salt. For example, the alkaline detergent comprises at least 10 mM of alkaline salt. For example, the alkaline detergent comprises at least 15 mM of alkaline salt. For example, the alkaline detergent comprises at least 20 mM of alkaline salt. For example, the alkaline detergent comprises at least 30 mM of alkaline salt. For example, the alkaline detergent comprises at least 40 mM of alkaline salt. For example, the alkaline detergent comprises at least 50 mM of alkaline salt. For example, the alkaline detergent comprises at least 60 mM of alkaline salt. For example, the alkaline detergent comprises at least 70 mM of alkaline salt. For example, the alkaline detergent comprises at least 80 mM of alkaline salt. For example, the alkaline detergent comprises at least 90 mM of alkaline salt. For example, the alkaline detergent comprises at least 100 mM of alkaline salt. For example, the alkaline detergent comprises at least 110 mM of alkaline salt. For example, the alkaline detergent comprises at least 120 mM of alkaline salt. For example, the alkaline detergent comprises at least 130 mM of alkaline salt. For example, the alkaline detergent comprises at least 140 mM of alkaline salt.

In one example, the alkaline detergent comprises 10 mM, 15 mM, 20 mM, 30 mM, 40 mM, 50 mM, 60 mM, 70 mM, 80 mM, or 90 mM of alkaline salt. In one example, the alkaline detergent comprises 10 mM, or 15 mM, or 20 mM, or 25 mM, or 30 mM, or 35 mM, or 40 mM, or 45 mM, or 50 mM, or 55 mM, or 60 mM, or 65 mM, or 70 mM, or 75 mM, or 80 mM, or 85 mM, or 90 mM, 95 mM, or 100 mM, or 105 mM, or 110 mM, or 115 mM, or 120 mM, or 125 mM, or 130 mM, or 135 mM, or 140 mM, or 145 mM, or 150 mM of alkaline salt. For example, the alkaline detergent comprises 10 mM of alkaline salt. For example, the alkaline detergent comprises 15 mM of alkaline salt. For example, the alkaline detergent comprises 20 mM of alkaline salt. For example, the alkaline detergent comprises 25 mM of alkaline salt. For example, the alkaline detergent comprises 30 mM of alkaline salt. For example, the alkaline detergent comprises 35 mM of alkaline salt. For example, the alkaline detergent comprises 40 mM of alkaline salt. For example, the alkaline detergent comprises 45 mM of alkaline salt. For example, the alkaline detergent comprises 50 mM of alkaline salt. For example, the alkaline detergent comprises 55 mM of alkaline salt. For example, the alkaline detergent comprises 60 mM of alkaline salt. For example, the alkaline detergent comprises 65 mM of alkaline salt. For example, the alkaline detergent comprises 70 mM of alkaline salt. For example, the alkaline detergent comprises 75 mM of alkaline salt. For example, the alkaline detergent comprises 80 mM of alkaline salt. For example, the alkaline detergent comprises 85 mM of alkaline salt. For example, the alkaline detergent comprises 90 mM of alkaline salt. For example, the alkaline detergent comprises 95 mM of alkaline salt. For example, the alkaline detergent comprises 100 mM of alkaline salt. For example, the alkaline detergent comprises 105 mM of alkaline salt. For example, the alkaline detergent comprises 110 mM of alkaline salt. For example, the alkaline detergent comprises 115 mM of alkaline salt. For example, the alkaline detergent comprises 120 mM of alkaline salt. For example, the alkaline detergent comprises 125 mM of alkaline salt. For example, the alkaline detergent comprises 130 mM of alkaline salt. For example, the alkaline detergent comprises 135 mM of alkaline salt. For example, the alkaline detergent comprises 140 mM of alkaline salt. For example, the alkaline detergent comprises 145 mM of alkaline salt. For example, the alkaline detergent comprises 150 mM of alkaline salt.

In one example, the one or more surfactants is selected from the group consisting of amine ethoxylates, amphocarboxylates, triethanolamine, Octyl-β-Dglucopyranoside (OGP), Polysorbate, Poloxamer, Lauryldimethyl-amine (LDAO), Myristyldimethylamine-N-oxide (TDAO), Sodium-cholate, Decyl-ß-D-glucopyranoside and Dodecyl maltoside. For example, the one or more surfactants is amine ethoxylates. For example, the one or more surfactants is amphocarboxylates. For example, the one or more surfactants is triethanolamine. For example, the one or more surfactants is OGP (Octyl-β-Dglucopyranoside). For example, the one or more surfactants is Polysorbate 80. For example, the one or more surfactants is Polysorbate 20. For example, the one or more surfactants is LDAO (Lauryldimethyl-amine. For example, one or more surfactants is TDAO (Myristyldimethylamine-N-oxide). For example, the one or more surfactants is Sodium-cholate. For example, the one or more surfactants is Decyl-ß-D-glucopyranoside. For example, the one or more surfactants is Dodecyl maltoside.

In one example, the alkaline detergent comprises between 0.01% and 10% (w/v) of the one or more surfactants. For example, the alkaline detergent comprises between 0.01% and 5% (w/v) of the one or more surfactants. In one example, the alkaline detergent comprises between 0.01% and 1.0% (w/v) of the one or more surfactants. For example, the alkaline detergent comprises between 0.01% and 1% (w/v) or 0.01% and 0.5% (w/v), or 0.01% and 0.1% (w/v), or 0.01% and 0.05% (w/v) of the one or more surfactants. For example, the alkaline detergent comprises about 0.02% (w/v) of the one or more surfactants. In one example, the alkaline detergent comprises between 0.5% and 1% (w/v) of the one or more surfactants.

In one example, the alkaline detergent comprises between 0.01% and 0.10% (w/v) of the one or more surfactants. For example, the alkaline detergent comprises between 0.01% and 0.05% or 0.05% and 0.1% (w/v) of the one or more surfactants. In one example, the alkaline detergent comprises between 0.10% and 0.15%, or 0.15% and 0.20%, or 0.20% and 0.25%, or 0.25% and 0.30%, or 0.30% and 0.35%, or 0.35% and 0.40%, or 0.40% and 0.45%, or 0.45% and 0.50%, or 0.50% and 0.55%, or 0.55% and 0.60%, or 0.60% and 0.65%, or 0.65% and 0.70%, or 0.70% and 0.75%, or 0.75% and 0.80%, or 0.80% and 0.85%, or 0.85% and 0.90%, or 0.90% and 0.95%, or 0.95% and 1.00%, or 1.00% and 1.05%, or 1.05% and 1.10%, or 1.10% and 1.20% (w/v) of the one or more surfactants. For example, the alkaline detergent comprises between 0.10% and 0.15% (w/v) of the one or more surfactants. For example, the alkaline detergent comprises between 0.15% and 0.20% (w/v) of the one or more surfactants. For example, the alkaline detergent comprises between 0.20% and 0.25% (w/v) of the one or more surfactants. For example, the alkaline detergent comprises between 0.25% and 0.30% (w/v) of the one or more surfactants. For example, the alkaline detergent comprises between 0.30% and 0.35% (w/v) of the one or more surfactants. For example, the alkaline detergent comprises between 0.35% and 0.40% (w/v) of the one or more surfactants. For example, the alkaline detergent comprises between 0.40% and 0.45% (w/v) of the one or more surfactants. For example, the alkaline detergent comprises between 0.45% and 0.50% (w/v) of the one or more surfactants. For example, the alkaline detergent comprises between 0.50% and 0.55% (w/v) of the one or more surfactants. For example, the alkaline detergent comprises between 0.55% and 0.60% (w/v) of the one or more surfactants. For example, the alkaline detergent comprises between 0.60% and 0.65% (w/v) of the one or more surfactants. For example, the alkaline detergent comprises between 0.65% and 0.70% (w/v) of the one or more surfactants. For example, the alkaline detergent comprises between 0.70% and 0.75% (w/v) of the one or more surfactants. For example, the alkaline detergent comprises between 0.75% and 0.80% (w/v) of the one or more surfactants. For example, the alkaline detergent comprises between 0.80% and 0.85% (w/v) of the one or more surfactants. For example, the alkaline detergent comprises between 0.85% and 0.90% (w/v) of the one or more surfactants. For example, the alkaline detergent comprises between 0.90% and 0.95% (w/v) of the one or more surfactants. For example, the alkaline detergent comprises between 0.95% and 1.00% (w/v) of the one or more surfactants. For example, the alkaline detergent comprises between 1.00% and 1.05% (w/v) of the one or more detergents. For example, the alkaline detergent comprises between 1.05% and 1.10% (w/v) of the one or more surfactants. For example, the alkaline detergent comprises between 1.10% and 1.20% (w/v) of the one or more surfactants.

In one example, the alkaline detergent comprises at least 0.01%, 0.02%, 0.03%, 0.04%, 0.05%, 0.06%, 0.07%, 0.08%, 0.09% (w/v) of the one or more surfactants. In one example, the alkaline detergent comprises at least 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.00%, 1.05%, 1.10%, or 1.20% (w/v) of the one or more surfactants. For example, the alkaline detergent comprises at least 0.01% (w/v) of the one or more surfactants. For example, the alkaline detergent comprises at least 0.02% (w/v) of the one or more surfactants. For example, the alkaline detergent comprises at least 0.03% (w/v) of the one or more surfactants. For example, the alkaline detergent comprises at least 0.04% (w/v) of the one or more surfactants. For example, the alkaline detergent comprises at least 0.05% (w/v) of the one or more surfactants. For example, the alkaline detergent comprises at least 0.06% (w/v) of the one or more surfactants. For example, the alkaline detergent comprises at least 0.07% (w/v) of the one or more surfactants. For example, the alkaline detergent comprises at least 0.08% (w/v) of the one or more surfactants. For example, the alkaline detergent comprises at least 0.09% (w/v) of the one or more surfactants. For example, the alkaline detergent comprises at least 0.10% (w/v) of the one or more surfactants. For example, the alkaline detergent comprises at least 0.15% (w/v) of the one or more surfactants. For example, the alkaline detergent comprises at least 0.20% (w/v) of the one or more surfactants. For example, the alkaline detergent comprises at least 0.25% (w/v) of the one or more surfactants. For example, the alkaline detergent comprises at least 0.30% (w/v) of the one or more surfactants. For example, the alkaline detergent comprises at least 0.35% (w/v) of the one or more surfactants. For example, the alkaline detergent comprises at least 0.40% (w/v) of the one or more surfactants. For example, the alkaline detergent comprises at least 0.45% (w/v) of the one or more surfactants. For example, the alkaline detergent comprises at least 0.50%, (w/v) of the one or more surfactants. For example, the alkaline detergent comprises at least 0.55% (w/v) of the one or more surfactants. For example, the alkaline detergent comprises at least 0.60% (w/v) of the one or more surfactants. For example, the alkaline detergent comprises at least 0.65% (w/v) of the one or more surfactants. For example, the alkaline detergent comprises at least 0.70% (w/v) of the one or more surfactants. For example, the alkaline detergent comprises at least 0.75% (w/v) of the one or more surfactants. For example, the alkaline detergent comprises at least 0.80% (w/v) of the one or more surfactants. For example, the alkaline detergent comprises at least 0.85% (w/v) of the one or more surfactants. For example, the alkaline detergent comprises at least 0.90% (w/v) of the one or more surfactants. For example, the alkaline detergent comprises at least 0.95% (w/v) of the one or more surfactants. For example, the alkaline detergent comprises at least 1.00% (w/v) of the one or more surfactants. For example, the alkaline detergent comprises at least 1.05% (w/v) of the one or more surfactants. For example, the alkaline detergent comprises at least 1.10% (w/v) of the one or more surfactants. For example, the alkaline detergent comprises at least 1.20% (w/v) of the one or more surfactants.

In one example, the alkaline detergent comprises 0.01%, 0.02%, 0.03%, 0.04%, 0.05%, 0.06%, 0.07%, 0.08%, 0.09% (w/v) of the one or more surfactants. In one example, the alkaline detergent comprises 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.00%, 1.05%, 1.10%, or 1.20% (w/v) of the one or more surfactants. For example, the alkaline detergent comprises 0.01% (w/v) of the one or more surfactants. For example, the alkaline detergent comprises 0.02% (w/v) of the one or more surfactants. For example, the alkaline detergent comprises 0.03% (w/v) of the one or more surfactants. For example, the alkaline detergent comprises 0.04% (w/v) of the one or more surfactants. For example, the alkaline detergent comprises 0.05% (w/v) of the one or more surfactants. For example, the alkaline detergent comprises 0.06% (w/v) of the one or more surfactants. For example, the alkaline detergent comprises 0.07% (w/v) of the one or more surfactants. For example, the alkaline detergent comprises 0.08% (w/v) of the one or more surfactants. For example, the alkaline detergent comprises 0.09% (w/v) of the one or more surfactants. For example, the alkaline detergent comprises 0.10% (w/v) of the one or more surfactants. For example, the alkaline detergent comprises 0.15% (w/v) of the one or more surfactants. For example, the alkaline detergent comprises 0.20% (w/v) of the one or more surfactants. For example, the alkaline detergent comprises 0.25% (w/v) of the one or more surfactants. For example, the alkaline detergent comprises 0.30% (w/v) of the one or more surfactants. For example, the alkaline detergent comprises 0.35% (w/v) of the one or more surfactants. For example, the alkaline detergent comprises 0.40% (w/v) of the one or more surfactants. For example, the alkaline detergent comprises 0.45% (w/v) of the one or more surfactants. For example, the alkaline detergent comprises 0.50% (w/v) of the one or more surfactants. For example, the alkaline detergent comprises 0.55% (w/v) of the one or more surfactants. For example, the alkaline detergent comprises 0.60% (w/v) of the one or more surfactants. For example, the alkaline detergent comprises 0.65% (w/v) of the one or more surfactants. For example, the alkaline detergent comprises 0.70% (w/v) of the one or more surfactants. For example, the alkaline detergent comprises 0.75% (w/v) of the one or more surfactants. For example, the alkaline detergent comprises 0.80% (w/v) of the one or more surfactants. For example, the alkaline detergent comprises 0.85% (w/v) of the one or more surfactants. For example, the alkaline detergent comprises 0.90% (w/v) of the one or more surfactants. For example, the alkaline detergent comprises 0.95% (w/v) of the one or more surfactants. For example, the alkaline detergent comprises 1.00% (w/v) of the one or more surfactants. For example, the alkaline detergent comprises 1.05% (w/v) of the one or more surfactants. For example, the alkaline detergent comprises 1.10% (w/v) of the one or more surfactants. For example, the alkaline detergent comprises 1.20% (w/v) of the one or more surfactants.

In one example, the chelator is ethylenediaminetetraacetic acid (EDTA), ethylene glycol-bis(β-aminoethyl ether)-N,N,N′,N′-tetraacetic acid (EGTA), sodium gluconate, trisodium citrate, diethylenetriaminepentaacetic acid (DTPA), meso-2,3-dimercaptosuccinic acid (DMSA), 2,3 dimercaptopropane-1-sulfonic acid (DMPS), 1,2-bis(o-aminophenoxy)ethane-N,N,N′,N′-tetraacetic acid (BAPTA), alpha-lipoic acid (ALA), ethylenediamine-N,N′-disuccinic acid (EDDS), iminodisuccinic acid (IDS), methylglycinediacetic acid (MGDA), triethylenetetramine (Trien), iminodiacetic acid (IDA), nitrilotriacetic acid (NTA), tripolyphosphate (TPP), sodium diethyldithiocarbamate (DDC), L-Glutamic acid N,N-diacetic acid (GLDA), tetrasodium salt, penicillamine, or of any salts thereof including a calcium or sodium salt thereof. For example, the chelator is ethylenediaminetetraacetic acid (EDTA). For example, the chelator is ethylene glycol-bis(β-aminoethyl ether)-N,N,N′,N′-tetraacetic acid (EGTA). For example, the chelator is sodium gluconate. For example, the chelator is trisodium citrate. For example, the chelator is diethylenetriaminepentaacetic acid (DTPA). For example, the chelator is meso-2,3-dimercaptosuccinic acid (DMSA). For example, the chelator is 2,3 dimercaptopropane-1-sulfonic acid (DMPS). For example, the chelator is 1,2-bis(o-aminophenoxy)ethane-N,N,N′,N′-tetraacetic acid (BAPTA). For example, the chelator is alpha-lipoic acid (ALA). For example, the chelator is ethylenediamine-N,N′-disuccinic acid (EDDS). For example, the chelator is iminodisuccinic acid (IDS). For example, the chelator is methylglycinediacetic acid (MGDA). For example, the chelator is triethylenetetramine (Trien). For example, the chelator is iminodiacetic acid (IDA). For example, the chelator is nitrilotriacetic acid (NTA). For example, the chelator is tripolyphosphate (TPP). For example, the chelator is sodium diethyldithiocarbamate (DDC). For example, the chelator is L-Glutamic acid N,N-diacetic acid (GLDA). For example, the chelator is tetrasodium salt. For example, the chelator is penicillamine.

In one example, the chelator is EDTA or sodium gluconate.

In one example, the chelator is a biodegradable chelator.

In one example, the biodegradable chelator is selected from a group consisting of ethylenediamine-N,N′-disuccinic acid (EDDS), iminodisuccinic acid (IDS), methylglycinediacetic acid (MGDA), and nitrilotriacetic acid (NTA).

In one example, the alkaline detergent comprises between 0.5% and 1%, or 1% and 1.5%, or 1.5% and 2.0%, or 2.0% and 2.5%, or 2.5% and 3%, or 3% and 3.5%, or 3.5% and 4%, or 4% and 4.5%, or 4.5% and 5%, or 5% and 5.5%, or 5.5% and 6%, or 6% and 6.5%, or 6.5% and 7.0%, or 7.0% and 7.5%, or 7.5% and 8.0%, or 8.0% and 8.5% (w/v) of a chelator. For example, the alkaline detergent comprises between 0.5% and 1% (w/v) of a chelator. For example, the alkaline detergent comprises between 1% and 1.5% (w/v) of a chelator. For example, the alkaline detergent comprises between 1.5% and 2.0% (w/v) of a chelator. For example, the alkaline detergent comprises between 2.0% and 2.5% (w/v) of a chelator. For example, the alkaline detergent comprises between 2.5% and 3% (w/v) of a chelator. For example, the alkaline detergent comprises between 3% and 3.5% (w/v) of a chelator. For example, the alkaline detergent comprises between 3.5% and 4% (w/v) of a chelator. For example, the alkaline detergent comprises between 4% and 4.5% (w/v) of a chelator. For example, the alkaline detergent comprises between 4.5% and 5% (w/v) of a chelator. For example, the alkaline detergent comprises between 5% and 5.5% (w/v) of a chelator. For example, the alkaline detergent comprises between 5.5% and 6% (w/v) of a chelator. For example, the alkaline detergent comprises between 6% and 6.5% (w/v) of a chelator. For example, the alkaline detergent comprises between 6.5% and 7.0% (w/v) of a chelator. For example, the alkaline detergent comprises between 7.0% and 7.5% (w/v) of a chelator. For example, the alkaline detergent comprises between 7.5% and 8.0% (w/v) of a chelator. For example, the alkaline detergent comprises between 8.0% and 8.5% (w/v) of a chelator.

In one example, the alkaline detergent comprises at least 0.5%, 1%, 1.5%, 2%, 2.5%, 3%, 3.5%, 4%, 4.5%, 5%, 5.5%, 6%, 6.5%, 7%, 7.5%, 8%, 8.5% (w/v) of a chelator. For example, the alkaline detergent comprises at least 0.5% (w/v) of a chelator. For example, the alkaline detergent comprises at least 1% (w/v) of a chelator. For example, the alkaline detergent comprises at least 1.5% (w/v) of a chelator. For example, the alkaline detergent comprises at least 2% (w/v) of a chelator. For example, the alkaline detergent comprises at least 2.5% (w/v) of a chelator. For example, the alkaline detergent comprises at least 3% (w/v) of a chelator. For example, the alkaline detergent comprises at least 3.5% (w/v) of a chelator. For example, the alkaline detergent comprises at least 4% (w/v) of a chelator. For example, the alkaline detergent comprises at least 4.5% (w/v) of a chelator. For example, the alkaline detergent comprises at least 5% (w/v) of a chelator. For example, the alkaline detergent comprises at least 5.5% (w/v) of a chelator. For example, the alkaline detergent comprises at least 6% (w/v) of a chelator. For example, the alkaline detergent comprises at least 6.5% (w/v) of a chelator. For example, the alkaline detergent comprises at least 7% (w/v) of a chelator. For example, the alkaline detergent comprises at least 7.5% (w/v) of a chelator. For example, the alkaline detergent comprises at least 8% (w/v) of a chelator. For example, the alkaline detergent comprises at least 8.5% (w/v) of a chelator.

In one example, the alkaline detergent comprises 0.5%, 1%, 1.5%, 2%, 2.5%, 3%, 3.5%, 4%, 4.5%, 5%, 5.5%, 6%, 6.5%, 7%, 7.5%, 8%, 8.5% (w/v) of a chelator. For example, the alkaline detergent comprises 0.5% (w/v) of a chelator. For example, the alkaline detergent comprises 1% (w/v) of a chelator. For example, the alkaline detergent comprises 1.5% (w/v) of a chelator. For example, the alkaline detergent comprises 2% (w/v) of a chelator. For example, the alkaline detergent comprises 2.5% (w/v) of a chelator. For example, the alkaline detergent comprises 3% (w/v) of a chelator. For example, the alkaline detergent comprises 3.5% (w/v) of a chelator. For example, the alkaline detergent comprises 4% (w/v) of a chelator. For example, the alkaline detergent comprises 4.5% (w/v) of a chelator. For example, the alkaline detergent comprises 5% (w/v) of a chelator. For example, the alkaline detergent comprises 5.5% (w/v) of a chelator. For example, the alkaline detergent comprises 6% (w/v) of a chelator. For example, the alkaline detergent comprises 6.5% (w/v) of a chelator. For example, the alkaline detergent comprises 7% (w/v) of a chelator. For example, the alkaline detergent comprises 7.5% (w/v) of a chelator. For example, the alkaline detergent comprises 8% (w/v) of a chelator. For example, the alkaline detergent comprises 8.5% (w/v) of a chelator.

In one example, the alkaline detergent is diluted, e.g., into water. For example, a chromatography resin is contacted with a solution comprising 0.1 to 5% alkaline detergent, e.g., 0.5 to 4% or 1 to 3% alkaline detergent. In one example, the chromatography resin is contacted with a solution comprising 0.5% alkaline detergent. In another example, the chromatography resin is contacted with a solution comprising 1% alkaline detergent. For example, the chromatography resin is contacted with a solution comprising 2% alkaline detergent.

In one example, multiple column volumes of an alkaline detergent are passed through a chromatography medium to sanitize the medium. The alkaline detergent can be passed through the chromatography medium in downflow direction and/or upflow direction. For example, multiple column volumes are passed through the chromatography medium in upflow direction and then multiple column volumes are passed through the chromatography medium in downflow direction. For example, at least three or four or five or six column volumes are passed through the chromatography medium in upflow direction. For example, at least three or four or five or six column volumes are passed through the chromatography medium in downflow direction.

In one example, an alkaline detergent is contacted to the chromatography medium for at least 10 minutes to sanitize the medium. For example, an alkaline detergent is contacted to the chromatography medium for at least 15 minutes or 30 minutes or 40 minutes or 50 minutes or 60 minutes. In one example, the alkaline detergent is contacted to the chromatography medium for no more than 120 minutes. For example, an alkaline detergent is contacted to the chromatography medium for between 15 and 120 minutes or between 30 and 100 minutes or between 40 and 90 minutes or between 50 and 70 minutes. For example, an alkaline detergent is contacted to the chromatography medium for about 60 minutes.

### Regeneration

The present disclosure further provides a method of regenerating the chromatography medium which may be performed before or after sanitizing the chromatography medium. For example, the chromatography medium is regenerated before sanitization. Regeneration of the chromatography medium restores the binding capacity and/or properties of the chromatography medium to enable its use in one or more further chromatography cycles.

Suitable solutions for regenerating the chromatography medium will be apparent to the skilled person and/or described herein.

Methods for regenerating a chromatography medium are known in the art and will be apparent to the skilled person. For example, regenerating a chromatography medium includes contacting a chromatography medium with a solution including but not limited to, salt solutions (NaCl), caustic solutions up to 1.5 molar sodium hydroxide, solvents such as for example acetic acid, ethanol, isopropanol and acetonitrile, Tris or Tris EDTA. Regeneration methods also include contacting the medium with a solution comprising ethylene glycol, sodium chloride, sodium hydroxide or a combination sodium hydroxide and ethanol.

In some examples, regenerating a chromatography medium comprises contacting the medium with a combination of solutions. The combination of solutions can be mixed prior to contacting the medium or contacted to the medium consecutively, optionally separated by a wash, e.g., with water.

As will be apparent to the skilled artisan, a method for regenerating a chromatography medium can be dependent on the type of chromatography medium.

An exemplary method for regenerating a cation exchange chromatography medium includes contacting the medium with 1M sodium hydroxide or 1M NaCl or a combination of an amino acid such as arginine and NaCl chloride or a combination of alcohol and sodium hydroxide or a combination of alcohol and NaCl.

An exemplary method for regenerating an anion exchange chromatography medium includes contacting the medium with 1M sodium hydroxide or 1M NaCl or a combination of sodium hydroxide or NaCl with urea and, optionally, EDTA or citric acid.

An exemplary method for regenerating g a hydrophobic interaction chromatography medium or a mixed mode chromatography medium includes contacting the medium with citric acid (10-200 mM) or hydrochloric acid (10 mM) or sodium hydroxide (0.5-1M) or guanidine hydrochloride (6M) or urea (2-8M) or propanol (40%) or 20-100% ethanol or methanol or a combination of Tris, EDTA and NaCl.

An exemplary method for regenerating an affinity chromatography medium, e.g., a protein A chromatography medium, comprises contacting the medium with a combination of sodium hydroxide (10-50 mM) and NaCl (1M) or urea or guanidine hydrochloride or citric acid.

Exemplary methods for regenerating an affinity chromatography medium such as FcXP are included in the following paragraphs. These methods are applicable to other chromatography media.

In one example, the method comprises regenerating the chromatography medium by contacting the medium with a solution comprising sodium chloride, arginine, ethanol, a polymer such as ethylene glycol, sodium hydroxide or a combination sodium hydroxide and ethanol. For example, the method comprises regenerating the chromatography medium by contacting the medium with ethylene glycol, e.g., 20% or 40% ethylene glycol. For example, the method comprises regenerating the chromatography medium by contacting the medium with a solution comprising sodium hydroxide. For example, the method comprises regenerating the chromatography medium by contacting the medium with a combination of sodium hydroxide and ethanol. For example, the method comprises regenerating the chromatography medium by contacting the medium with ethanol.

In one example, the method comprises regenerating the chromatography medium by contacting the medium with ethanol and then contacting the resin with sodium hydroxide.

In one example, the resin is contacted with 5% to 50% ethanol, e.g., 10% to 40% ethanol, e.g., 15% to 30% ethanol. For example, the resin is contacted with 20% ethanol. In one example, the resin is contacted with 5% ethanol. In one example, the resin is contacted with 10% ethanol. In one example, the resin is contacted with 15% ethanol. In one example, the resin is contacted with 20% ethanol. In one example, the resin is contacted with 25% ethanol. In one example, the resin is contacted with 30% ethanol. In one example, the resin is contacted with 35% ethanol. In one example, the resin is contacted with 40% ethanol. In one example, the resin is contacted with 45% ethanol. In one example, the resin is contacted with 50% ethanol.

In one example, the resin is contacted with 5 mM to 50 mM sodium hydroxide, e.g., 10 mM to 40 mM sodium hydroxide, e.g., 15 mM to 30 mM sodium hydroxide. For example, the resin is contacted with 20 mM sodium hydroxide. In one example, the resin is contacted with 5 mM sodium hydroxide. In one example, the resin is contacted with 10 mM sodium hydroxide. In one example, the resin is contacted with 15 mM sodium hydroxide. In one example, the resin is contacted with 25 mM sodium hydroxide. In one example, the resin is contacted with 30 mM sodium hydroxide. In one example, the resin is contacted with 35 mM sodium hydroxide. In one example, the resin is contacted with 40 mM sodium hydroxide. In one example, the resin is contacted with 45 mM sodium hydroxide. In one example, the resin is contacted with 50 mM sodium hydroxide.

In one example, the medium is contacted with a solution comprising 20% ethanol and then a solution comprising 5 mM or 10 mM or 10 mM or 15 mM or 20 mM sodium hydroxide. In one example, the medium is contacted with a solution comprising 20% ethanol and then a solution comprising 5 mM sodium hydroxide. In one example, the medium is contacted with a solution comprising 20% ethanol and then a solution comprising 20 mM sodium hydroxide.

In one example, the ethanol and sodium hydroxide are included in the same solution. Suitable amounts or concentrations of ethanol and sodium hydroxide are described herein. For example, the medium is contacted with a solution comprising 20% ethanol and 5 mM sodium hydroxide. In one example, the medium is contacted with a solution comprising 20% ethanol and 20 mM sodium hydroxide.

In one example, a chromatography medium is regenerated using ethylene glycol. For example, the chromatography medium is contacted with a solution comprising 20% ethylene glycol and/or a solution comprising 40% ethylene glycol. For example, the medium is contacted with a solution comprising 20% ethylene glycol and then a solution comprising 40% ethylene glycol.

In one example, multiple column volumes of a solution for regenerating a chromatography medium are passed through a chromatography medium to regenerate the medium. The solution can be passed through the chromatography medium in downflow direction and/or upflow direction. For example, multiple column volumes are passed through the chromatography medium in upflow direction and then multiple column volumes are passed through the chromatography medium is downflow direction. For example, at least one or two or three or four or five or six column volumes are passed through the chromatography medium in upflow direction. For example, at least one or two or three or four or five or six column volumes are passed through the chromatography medium in downflow direction.

In one example, a method of the disclosure comprises:
(i) regenerating a chromatography medium as described herein; and
(ii) sanitizing the chromatography medium of (i) as described herein.

In one example, a method of the disclosure comprises:
(i) regenerating a chromatography medium by contacting the medium with a solution comprising ethanol and a solution comprising sodium hydroxide, e.g., as described herein; and
(ii) sanitizing the chromatography medium of (i) by contacting the medium with an alkaline detergent or a solution comprising same as described herein.

In one example, a method of the disclosure comprises:
(i) regenerating a chromatography medium by contacting the medium with a solution comprising ethanol and sodium hydroxide as described herein; and
(ii) sanitizing the chromatography medium of (i) by contacting the medium with an alkaline detergent or a solution comprising same as described herein.

An advantage of sanitizing and regenerating the chromatography medium using the method of the present disclosure is that the effective lifetime of the chromatography medium can be increased in comparison to conventional sanitization and/or regeneration methods. For example, the methods of the present disclosure can allow the chromatography medium to be used at least 1, 2, 3, 4, 5, 10, 25 or more times more often than compared to the chromatography medium not sanitized and/or regenerated using the method of the present disclosure. The addition of even 1 or 2 purification batches to the lifetime of the chromatography medium would provide an advantage, for example, a cost and/or time saving advantage in the purification of a desired protein from a sample.

### Continuous Chromatography

The present disclosure of sanitizing (and optionally regenerating) a chromatography medium may be performed as part of a process of continuous chromatography.

The term “continuous chromatography” shall be taken to mean a chromatographic method comprising one or more column(s) packed with a chromatography medium, wherein each column comprises one or more zones. A zone is a column, or a region of a column, comprising the chromatography medium where one or more chromatography steps can be performed. For example, a zone is selected from a group consisting of an equilibration zone, a binding zone, a wash zone, an elution zone, a stripping zone, or a combination thereof.

Continuous chromatography comprising more than one column involves the columns being connected in an arrangement that allows the columns to be operated in series and/or in parallel. In principle, a sample comprising a desired protein may be loaded on a first and/or subsequent columns while other columns (or other zones of a column) are going through an equilibration, wash, elution, sanitization and/or regeneration simultaneously. Examples of continuous chromatography will be apparent to the skilled person and/or described herein.

Examples of columns which may be used to perform the method of the present disclosure will be apparent to the skilled person and/or described herein. For example, the continuous chromatography method may be performed using Tricorn 5/100 columns (Cytiva) or Eco plus 1 cm columns (YMC). In another example, the continuous chromatography method may be performed using BioSMB PD System (Sartorius).

In one example, the continuous chromatography is continuous affinity chromatography.

The term “continuous affinity chromatography” shall be taken to mean a chromatographic method comprising one or more column(s) packed with identical affinity chromatography medium, wherein each column comprises one or more zones.

### Simulated Moving Bed (SMB) Chromatography

In one example, the continuous chromatography is simulated moving bed (SMB) chromatography. The term “simulated moving bed chromatography” or “SMB chromatography” refers to a chromatography method first described in U.S. Pat. No. 2,985,589. Examples of SMB chromatography setup and/or apparatus will be apparent to the skilled person and/or described herein. The concept of simulated moving bed involves the use of multiple smaller columns (rather than one large column) containing a solid absorbent (e.g. affinity chromatography medium) and performing one or more continuous chromatography steps (i.e. equilibration, binding, washing, eluting or stripping) simultaneously on different columns in a continuous loop.

An example of a SMB chromatography set up has columns arranged into four sections with one or more columns per section. Two inlet streams (feed and eluent) and two outlet streams (extract and raffinate) are directed in alternating order to and from the column ring. The inlet and outlet positions are switched at regular time intervals in the direction of the liquid flow, thus simulating counter-current movement of columns. A feed (containing adsorbable components (extract)) is loaded onto one or more columns of the SMB chromatography setup, and the extract binds to the chromatography medium within the columns. Meanwhile, less adsorbed components (raffinate) in the feed pass through the column. The raffinate may be loaded onto one or more subsequent column(s) or removed from the SMB chromatography system as waste. An eluent is loaded onto the column to collect the extract. For example, an eluate may be collected from a first column while more feed is loaded onto one or more subsequent column(s).

The chromatography medium in SMB chromatography may undergo multiple cycles (e.g. 50 cycles) of medium equilibration, loading, binding and/or elution of the protein of interest, along with stripping, sanitizing, and/or regeneration of the chromatography medium. Multiple batch runs (e.g. 4 to 10 batches) may be performed using SMB chromatography. The total lifetime of the chromatography medium in SMB chromatography can be in the range of 200 to 500 cycles (if not more) before the chromatography medium is unusable.

### Periodic Counter-Current Chromatography (PCC)

In one example, the continuous chromatography is periodic counter-current chromatography (PCC). Examples of PCC setup and/or apparatus will be apparent to the skilled person and/or described herein. The concept of PCC involves the use of multiple columns containing a solid absorbent (e.g. affinity chromatography medium) and performing the chromatography steps in parallel in a quasi-continuous manner.

An example of PCC setup involves the use of two columns. In a first step, a sample is loaded onto a first column above the DBC of the chromatography medium so that unbound product (e.g. IgG) breaks through the first column and is captured by the second column. In a second step, the first column is washed, eluted, cleaned and/or re-equilibrated independently of the second column being loaded with a further sample. In a third step, an additional sample is loaded onto the second column above the DBC of the chromatography medium so that unbound product breaks through the second column and is captured by the first column. In a fourth step, the second column is washed, eluted, cleaned and/or re-equilibrated independently of the first column being loaded with a further sample. The process steps are continuously cycled between the two columns.

Another example of PCC setup involves the use of multiple columns. For example, a variation of the above PCC setup can involve use of multiple columns to capture unbound product which simulates use of a large column.

### Continuous Counter-Current Tangential Chromatography (CCTC)

In one example, the continuous chromatography is continuous counter-current tangential chromatography (CCTC). Examples of CCTC setup and/or apparatus will be apparent to the skilled person and/or described herein. The concept of CCTC involves using the chromatography medium in a slurry form where the slurry is continuously directed through a number of static mixers and hollow fiber membranes which separate the fluid phase from the chromatography medium. CCTC is ordinarily performed at low pressures (e.g. <70 kPa).

An example of a CCTC process involves binding, first wash, second wash, elution, sanitization, regeneration and/or equilibration steps. Sample (e.g. plasma or fraction thereof) and the chromatography medium is passed through static mixers and hollow fiber membranes in a binding step. Impurities are removed in the flow through of the hollow fiber membranes in the washing step, while chromatography medium bound product is retained by the membrane. The hollow fibres retain the chromatography medium and allow the product to flow through in the elution step. The chromatography medium is sanitized, regenerated and/or equilibrated and process is repeated.

### Continuous Counter-Current Spiral Chromatography (CCSC)

In one example, the continuous chromatography is continuous counter-current spiral chromatography (CCSC). Examples of CCSC setup and/or apparatus will be apparent to the skilled person and/or described herein. The concept of CCSC involves the use of a compact rotating coil separation column mounted onto a centrifuge rotary frame. There are two separation column designs currently available: the spiral disk assembly and the spiral tube support assembly.

An exemplary CCSC process involves a coiled separation column revolving around a central axis of the centrifuge while it synchronously rotates about its own axis (at e.g., 1,000 to 1,200 rpm). A mobile phase can be passed through the centrifuge rotor without rotary seals, and a large amount of a stationary phase is retained while the two phases are mixed along the length of the column to produce a highly efficient solute separation.

### Analysis of the Chromatography Medium

### Sanitization

Methods of determining the sanitization of the chromatography medium will be apparent to the skilled person and/or described herein.

In one example, sanitization is determined by measuring the amount of microorganism before and after sanitization using microbial challenge test. Briefly, the chromatography medium is loaded with a microorganism (e.g. Pseudomonas aeruginosa) at greater than 10⁶ CFU/ml. The chromatography medium is sanitized using a method of the present disclosure, one or more samples after sanitization is collected, and the number of viable microorganism (CFU) per mL is determined.

In another example, sanitization is determined by measuring the amount of virus before and after sanitization using a virus spiking challenge. Briefly, a sample is spiked with a known quantity of a virus and the sample run through a chromatography matrix. The chromatography matrix is sanitized using a method of the present disclosure, and one or more samples collected after sanitization. The amount of virus after sanitization is determined and compared to the amount prior to sanitization. A reduction in the amount of virus, e.g., to levels below the level of detection of the assay indicates that the sanitization method is effective.

### Regeneration

Methods of determining the regeneration of the chromatography medium will be apparent to the skilled person and/or described herein.

In one example, regeneration is determined by assessing dynamic binding capacity of a resin using a known quantity of a pure protein of interest (e.g., IgG) before and after regeneration using a method of the disclosure. For example, a similar dynamic binding capacity is determined before and after regeneration.

In another example, regeneration is determined by monitoring the change in protein content of the chromatography medium using spectrophotometry. Briefly, the area under the UV (A280) peak is measured before, during and/or after the regeneration of the chromatography medium to determine the change in the level of proteins (e.g. host cell proteins or other protein-based contaminants) of the medium.

### EXAMPLES

### Example 1: Materials and Methods

### Samples

Cryo-rich plasma (CRP) was prepared from pooling blood donations and stored at −20° C. The CRP was thawed at 37° C., filtered through a 1.2 μm Sartopure® (Sartorius) and 0.45+0.2 μm Sartobran® P (Sartorius) capsule filters at a pressure below 0.5 bar, and provided at room temperature for use in a chromatography method described herein.

Samples used to determine binding capacity of the chromatography medium after sanitization and regeneration of a chromatography medium were prepared from an eluate containing IgG from a batch chromatography run of CRP on ÄKTA system. Prior to use in determining binding capacity of a chromatography medium, the volume of the solution containing IgG was adjusted with EQB buffer (20 mM sodium phosphate, 500 mM NaCl, pH 7.4) to an optical density (OD) of approximately 7, a pH of ˜7.4 and conductivity of ˜21 mS/cm and filtered through a 0.45 μm syringe filter (Sterives, SVGV010RS) and 0.22 μm syringe filter (Sterives, SVH010RS).

### Chromatography Medium

Affinity chromatography medium CaptureSelect® FcXP (Thermo Fisher) was used to assess the method described herein. The affinity chromatography medium was resuspended and packed in Tricorn columns from Cytiva (column volume 1.31 ml and column height 6.7 cm) under 20 ml/min flow with 0.1 M sodium chloride (NaCl) until the chromatography medium bed was formed. Affinity chromatography using the columns packed with CaptureSelect® FcXP (Thermo Fisher) was performed in simulated moving bed mode on a BioSMB PD system (Sartorius).

### Binding Capacity

In a lifetime study of the chromatography medium of the present disclosure (e.g. as described in Example 3), binding capacity of the chromatography medium was determined.

Breakthrough curve (BTC) measurements of the chromatography medium contacted with an EQB buffer (at the start of the lifetime study) or after regeneration and sanitization procedure was determined using the Äkta avant 25 system (Cytiva) at a UV absorbance of 280 nm.

Commercially available software (e.g. UNICORN Evaluation Classic) was used to calculate the binding capacity of the chromatography medium contacted with the EQB buffer or the alkaline detergent based on the BTC measurements. The binding capacity of the chromatography medium was expressed as a percentage (%) compared to the binding capacity of the chromatography medium at the start of a lifetime study (e.g. BTC measurement of the chromatography medium contacted with the EQB buffer).

### Yield

Concentration of IgG in FcXP eluate, column flow through, and waste fraction (EQB phase) following purification of immunoglobulin from plasma and subsequent regeneration and sanitization was determined using immunonephelometry. Briefly, the light scattering characteristics of IgG in test samples was determined and compared to the light scattering characteristics of a set of standard solutions comprising known concentrations of IgG. The yield was expressed as a percentage (%) relative to the IgG content in the starting material (i.e. sample loaded onto the chromatography medium).

### Immunoglobulin Class Distribution

Quantification of immunoglobulin subclass and albumin in a FcXP eluate following purification of immunoglobulin from plasma and subsequent regeneration and sanitization was determined using immunonephelometry. Briefly, the light scattering characteristics of immunoglobulin (i.e. IgG, IgM, IgA) and albumin in the FcXP eluate was determined compared to the light scattering characteristics of a set of standard solutions comprising known concentrations of specific immunoglobulin or albumin.

### Purity

Purity of the FcXP eluate following purification of immunoglobulin from plasma and subsequent regeneration and sanitization was determined by LabChip® electrophoresis (Perkin Elmer). The LabChip is based on traditional gel electrophoresis principles that have been transferred to a microfluidic chip format to ensure a high throughput analysis. The chip format dramatically reduces separation time from 90 minutes to ˜1 minute and can provides automated molecular weight size, purity, and concentration of protein samples in a digital format. For determination of purity of samples, Protein Exact Assay from Perkin Elmer (manufacturer) was utilized.

Briefly, the FcXP eluate was loaded into individual wells of a chip used in LabChip® electrophoresis (Perkin Elmer), and the chip loaded into a characterization system (e.g. LabChip GXII Touch protein characterization system) and purity determined using LabChip Reviewer software.

### SDS-PAGE Analysis

Purity of the FcXP eluate following purification of immunoglobulin from plasma and subsequent regeneration and sanitization was determined qualitatively by SDS-PAGE. Briefly, one or more FcXP eluate following purification of immunoglobulin from plasma and subsequent regeneration and sanitization described herein were loaded onto a suitable SDS-PAGE gel (e.g. 8-16% TRIS-glycine), along with a protein size marker and a positive control for IgG (e.g. Privigen) under reduced and non-reduced conditions. Proteins were separated based on size and bands visualised by Coomassie blue staining. Purity was determined by analysing the bands of FcXP eluate following purification of immunoglobulin from plasma and subsequent regeneration and sanitization compared to bands of the positive control for IgG (e.g. Privigen).

### Example 2: Identification of Suitable Regeneration and Sanitization Solutions

To identify a solution suitable for regeneration of FcXP chromatography medium following purification of immunoglobulin from serum or plasma, the following solutions were tested:
EQB buffer with 1% Tween 80
300 mM arginine
0.5% caprylate
20% ethanol
40% ethanol
20% and 40% ethylene glycol
20 mM sodium hydroxide
10 mM DTT

Results showed 20% ethanol, 20% or 40% ethylene glycol and 20 mM sodium hydroxide to be most effective for regenerating a FcXP resin.

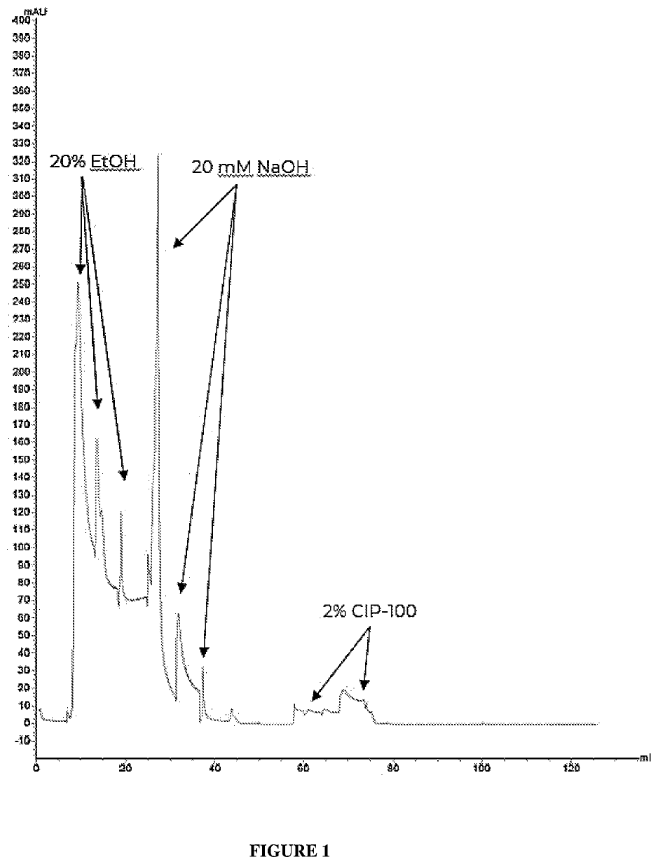

A sequence of 20% ethanol followed by 20 mM sodium hydroxide was then tested and shown to be effective for regenerating a FcXP resin. An exemplary chromatogram showing regeneration of the FcXP resin with 20% ethanol and 20 mM sodium hydroxide is shown in FIG. 1.

Analysis of the flow through from the FcXP chromatography medium during regeneration using reducing or non-reducing polyacrylamide gel electrophoresis showed that ethanol primarily removes the protein Clusterin whereas sodium hydroxide removes the remaining non-specifically bound proteins.

The alkaline detergent CIP-100 diluted to 2% was used for sanitization. Results showing sanitization of a FcXP chromatography media with 2% CIP-100 is also shown in FIG. 1.

### Example 3: FcXP Lifetime Studies

Studies were performed to determine the effects of 20% ethanol and 20 mM sodium hydroxide regeneration and 2% CIP-100 sanitization on FcXP lifetime. After each batch of continuous chromatography, the resin was exposed to following regeneration and sanitization conditions:

### 20% EtOH (Total Contact Time: 62 Minutes)

1) 5 CV, 1.3 ml/min, upflow
2) 4 CV, 0.1 ml/min, upflow
3) 5 CV, 1.3 ml/min, downflow

### 20 mM NaOH (Total Contact Time: 62 Minutes)

1) 5 CV, 1.3 ml/min, upflow
2) 4 CV, 0.1 ml/min, upflow
3) 5 CV, 1.3 ml/min, downflow

### 2% CIP-100 (Steps 2-5 Performed Over 61 Minutes)

1) EQB, 5 CV, 1.3 ml/min, upflow
2) CIP, 3 CV, 1.3 ml/min, upflow
3) CIP, 3 CV, 1.3 ml/min, downflow
4) CIP, 3 CV, 1.3 ml/min, upflow
5) CIP, 4 CV, 0.1 ml/min, upflow
6) EQB, 20 CV, 1.3 ml/min, upflow
7) EQB, 20 CV, 1.3 ml/min, downflow

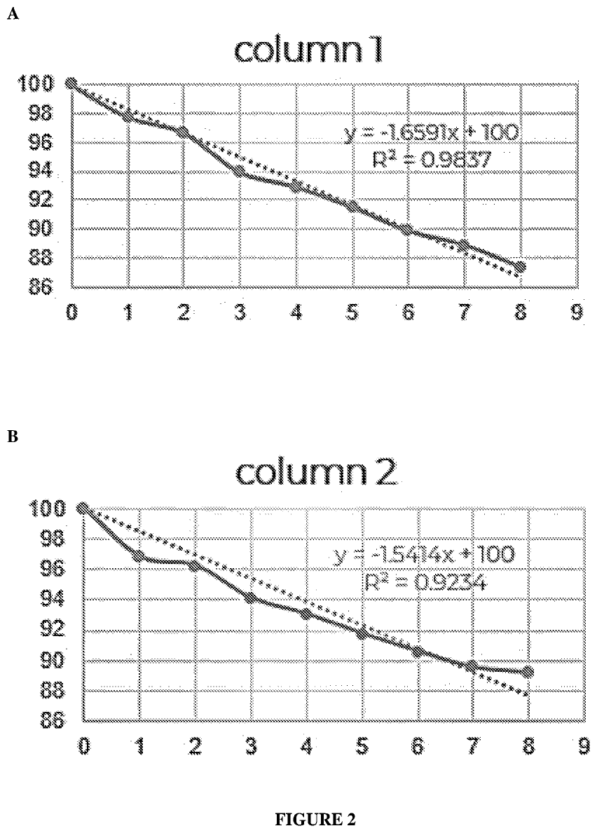

Table 1 and FIG. 2 shows the effect of the regeneration and sanitization on binding capacity of the FcXP chromatography medium over eight batches of purification of immunoglobulin from plasma.

TABLE 1
Dynamic binding capacity determination of FcXP chromatography medium
over multiple cycles of purification, regeneration and sanitization.

| | Fresh | After | After | After | After | After | After | After | After |
|---|---|---|---|---|---|---|---|---|---|
| | resin | Batch 1 | Batch 2 | Batch 3 | Batch 4 | Batch 5 | Batch 6 | Batch 7 | Batch 8 |
| column 1 | 100.0 | 97.8 | 96.7 | 93.9 | 92.9 | 91.5 | 89.8 | 88.9 | 87.3 |
| (%) | | | | | | | | | |
| column 2 | 100.0 | 96.8 | 96.2 | 94.1 | 93.0 | 91.7 | 90.6 | 89.5 | 89.2 |
| (%) | | | | | | | | | |

The data in Table 1 and FIG. 2 show a small decrease in binding capacity of about 1.6% per batch and regeneration/sanitization cycle.

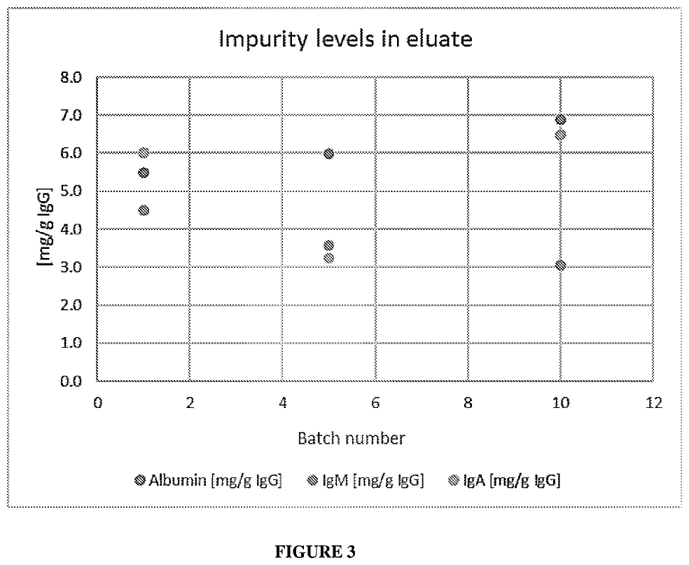

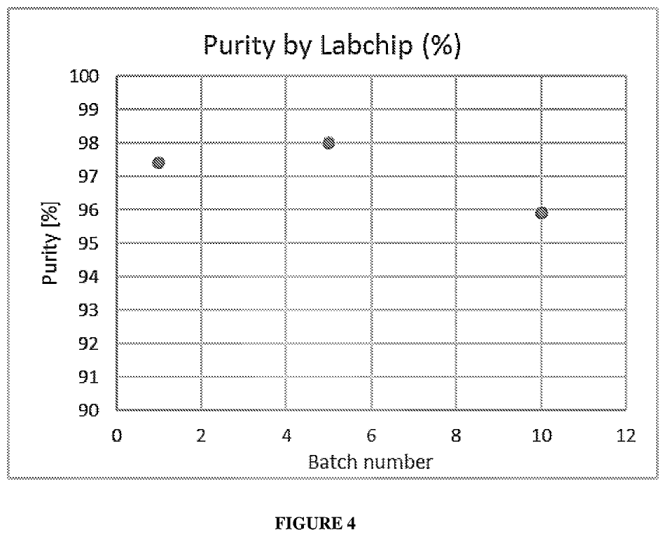

Table 2 and FIGS. 3-4 show analytics of purification of immunoglobulin from 10 purification batches from two pools of plasma (pool A derived from 25 donors and pool B derived from 10 donors). The recovered immunoglobulin was also generally greater than 95% pure. The data from BCA assay measured in blank run eluate additionally show that the regeneration sequence is effective in avoiding protein carryover.

TABLE 2
Analysis of eluates from 1^{st}, 5^{th} and 10^{th} batch of
purification of immunoglobulin from plasma and
subsequent regeneration and sanitization.

| | Batch 1 | Batch 5 | Batch 10 |
|---|---|---|---|
| Plasma pool | A | B | A |
| IgG in plasma pool/ | 6.76 | 7.56 | 6.88 |
| starting material (g/L) | | | |
| IgG Yield in FcXP | 98.4 | 93.8 | 95.5 |
| eluate (%) | | | |
| Total recovery in | 99.2 | 97.9 | 97.1 |
| all fractions* (%) | | | |
| Purity Labchip (%) | 97.43 | 98.03 | 95.93 |
| IgM/IgG (mg/g) | 4.5 | 3.6 | 3.1 |
| IgA/IgG (mg/g) | 6.0 | 3.2 | 6.5 |
| Albumin/IgG (mg/g) | 5.5 | 6.0 | 6.9 |
| Blank run BCA (mg/L) | <0.33 | <0.33 | Not |
| | | | executed |

*Total IgG recovery is calculated from total IgG amounts measured in all output fractions collected from the SMB device in relation to IgG amounts in starting material
**Estimate of protein carry-over

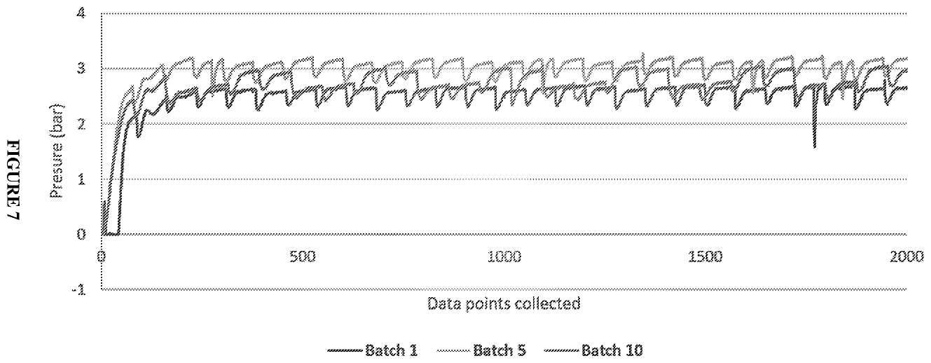

FIG. 7 shows the effect of the regeneration and sanitization on pressure profile of the FcXP chromatography medium over ten batches of purification of immunoglobulin from plasma, wherein regeneration and sanitization was performed after each batch (1 batch=50 product cycles).

### Example 4: CIP-100 as a Regeneration and Sanitization Solution

Experiments were conducted to compare the efficiency of 2% CIP-100 alone as a regeneration and sanitization solution compared to 20% ethanol 20 mM sodium hydroxide for regeneration and 2% CIP-100 for sanitization. Results showed that the decrease in protein binding capacity of the resin was similar for both strategies for regeneration/sanitization, however there was a slightly higher pressure increase when using CIP-100 alone (15.7%) compared to ethanol/sodium hydroxide/CIP-100 (11.2%). Estimated protein carry over was similar for both strategies, however SDS-PAGE indicated that slightly more protein remained bound to the resin after regeneration/sanitization using CIP-100 alone.

### Example 5: Optimization of Regeneration

To optimize the regeneration of FcXP chromatography medium following purification of immunoglobulin from plasma, studies are performed to study the effect of reducing the CV for each regeneration step.

Additionally, studies are performed to assess the effect of combining ethanol and sodium hydroxide into a single solution. These studies are performed with a constant 20% ethanol and varying the concentration of sodium hydroxide from 5 mM to 20 mM.

### Example 6: Application of Regeneration and Sanitization to Additional Chromatography Media

Affinity chromatography resins MabSelect® SuRe and MabSelect® SuRe LX (Thermo Fisher) are used to evaluate the suitability of alkaline detergents in the sanitization of chromatography media. Each chromatography medium is packed in Tricorn columns from Cytiva (diameter 5 mm) and chromatography is performed on an Äkta avant 25 system (Cytiva). Following one or more chromatography cycles in the purification of a protein of interest, the microbial load of the chromatography medium is determined.

Each chromatography medium is then regenerated using 20% ethanol followed by 20 mM sodium hydroxide and then sanitized by contacting the medium with an alkaline detergent (between 0.5% to 4.5%). The alkaline detergent used for sanitization is CIP-100 (Steris Life Sciences) at a concentration of 0.5%, 2% and 4.5%.

### Example 7: Assessment of Additional Alkaline Detergents

The sanitization and regeneration of the chromatography media as detailed in Examples 2 and 5 are repeated using additional alkaline detergents. The regeneration step is maintained as 20% ethanol followed by 20 mM sodium hydroxide. Specifically, alkaline detergents ProKlenz One (Steris Life Sciences), ProKlenz-100 (Steris Life Sciences) and COSA CIP-92 (Ecolab) at a concentration of concentration of 0.5%, 2% and 4.5% are tested, and alkaline detergents comprising potassium hydroxide (alkaline salt), EDTA (chelating agent) and a surfactant is tested.

### Example 8: Virus Inactivation by CIP-100

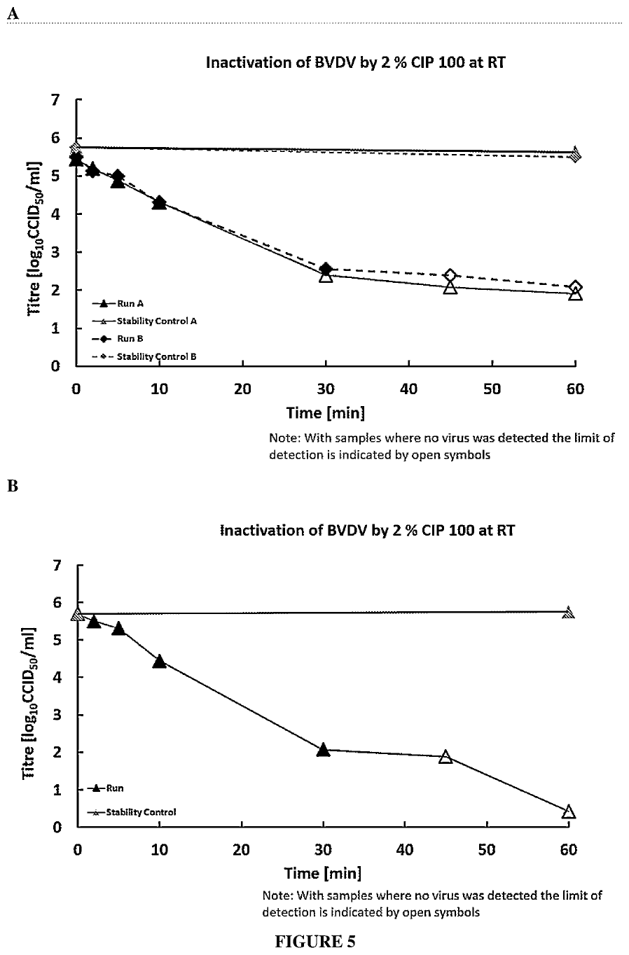

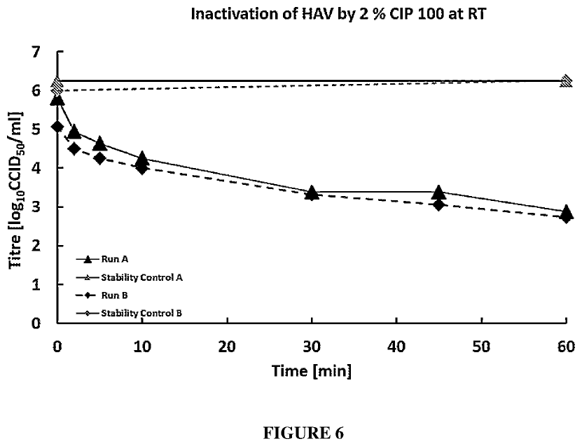

To test the efficacy of CIP-100 for viral inactivation, samples spiked with either Bovine Viral Diarrhea Virus (BVDV; a model for hepatitis C virus) or hepatitis A virus (HAV) were loaded onto a FcXP resin. Chromatography was performed and the column sanitized with 2% CIP-100. As shown in FIGS. 5 and 6, CIP-100 inactivates BVDV to levels below the limit of detection (i.e., complete inactivation) and inactivates HAV in the order of 3 logs.

### Example 9: Virus and Prion Inactivation by CIP-100

To test the efficacy of CIP-100 for viral and prion inactivation, samples spiked with virus or prion are loaded onto a FcXP resin. Chromatography is performed and the column is sanitized with 2% CIP-100. The eluent is then subject to a second, unspiked chromatography and eluent analyzed. If samples from the second chromatography are free of infectious virus or prions or levels are substantially reduced, the sanitization is effective.

1. A method of sanitizing a chromatography medium, the method comprising contacting the medium with an alkaline detergent.

2. The method of claim 1, wherein the alkaline detergent comprises an alkaline salt, one or more surfactants and, optionally, a chelator.

3. The method of claim 2, wherein the alkaline salt is selected from the group consisting of potassium hydroxide, sodium hydroxide, sodium carbonate, sodium acetate, sodium sulphide, and sodium bicarbonate, for example, the alkaline salt is potassium hydroxide or sodium hydroxide.

4. The method of claim 2, wherein the one or more surfactants is selected from the group consisting of amine ethoxylates, amphocarboxylates, triethanolamine, Octyl-β-Dglucopyranoside (OGP), Polysorbate, Poloxamer, Lauryldimethyl-amine (LDAO), Myristyldimethylamine-N-oxide (TDAO), Sodium-cholate, Decyl-ß-D-glucopyranoside and Dodecyl maltoside.

5. The method of claim 2, wherein the chelator is ethylenediaminetetraacetic acid (EDTA), ethylene glycol-bis(β-aminoethyl ether)-N,N,N′,N′-tetraacetic acid (EGTA), ethylenediamine-N,N′-disuccinic acid (EDDS), iminodisuccinic acid (IDS), methylglycinediacetic acid (MGDA), triethylenetetramine (Trien), iminodiacetic acid (IDA), nitrilotriacetic acid (NTA), tripolyphosphate (TPP), diethylenetriaminepentaacetic acid (DTPA), sodium diethyldithiocarbamate (DDC), L-Glutamic acid N,N-diacetic acid, tetrasodium salt (GLDA), and penicillamine, sodium gluconate, trisodium citrate, DMSA, DMPS, BAPTA, ALA and a salt thereof, for example, the chelator is a biodegradable chelator, such as a biodegradable chelator selected from the group consisting of ethylenediamine-N,N′-disuccinic acid (EDDS), iminodisuccinic acid (IDS), methylglycinediacetic acid (MGDA), nitrilotriacetic acid (NTA), and a salt thereof.

6. The method of claim 1, wherein the alkaline detergent comprises:
(i) between 0.01% to 10% (w/v) of one or more surfactants;
(ii) between 0.01% to 1% (w/v) of one or more surfactants;
(iii) between 10 mM and 150 mM of an alkaline salt; and/or
(iv) between 50 mM and 100 mM of an alkaline salt.

7. (canceled)

8. (canceled)

9. (canceled)

10. The method of claim 2, wherein the alkaline salt is at a concentration of 75 mM.

11. The method of claim 1, wherein the alkaline detergent comprises:
(i) TDAO and an alkaline salt selected from sodium hydroxide and potassium hydroxide;
(ii) between 10 mM to 150 mM of potassium hydroxide or sodium hydroxide and between 0.01% to 0.1% (w/v) of TDAO; or
(iii) 75 mM of potassium hydroxide or sodium hydroxide and 0.02% (w/v) of TDAO.

12. (canceled)

13. (canceled)

14. The method of claim 1, wherein the method comprises contacting the medium with the alkaline detergent for 60 minutes.

15. The method of claim 1, additionally comprising regenerating the chromatography medium, for example, regenerating the chromatography medium prior to sanitizing the chromatography medium.

16. The method of claim 15, comprising regenerating the chromatography medium by contacting the medium with a solution comprising ethylene glycol, sodium chloride, sodium hydroxide or a combination of sodium hydroxide and ethanol.

17. The method of claim 1, wherein the chromatography medium:
(i) is an ion exchange medium, an affinity medium, a hydrophobic interaction chromatography medium, a size exclusion column or a mixed mode chromatography medium;
(ii) comprises a matrix selected from the group consisting of poly(styrene-divinylbenzene), cross-linked poly(styrene-divinylbenzene), silica, agarose, cross-linked agarose, controlled pore glass, polymethacrylate and cellulose, for example, the chromatography medium comprises cross-linked poly(styrene-divinylbenzene), for example, the chromatography medium comprises a VHH antibody fragment capable of specifically binding to a CH3 domain of human IgG conjugated to a cross-linked poly(styrene-divinylbenzene) matrix; and/or
(ii) has been previously contacted with a sample, for example, the sample is plasma or a fraction thereof.

18. The method of claim 17, wherein the medium is an affinity chromatography medium.

19. The method of claim 18, wherein the affinity chromatography medium binds to an Fc region of an antibody, for example, the affinity chromatography medium comprises protein A or an antibody binding fragment thereof, protein G or an antibody binding fragment thereof or a ligand capable of specifically binding to a CH1 or a CH3 domain of human IgG, for example, the ligand capable of specifically binding to a CH3 domain of human IgG comprises a camelid-derived single domain [VHH] antibody fragment.

20. (canceled)

21. The method of claim 1, comprising:
(i) contacting a chromatography medium comprising a VHH antibody fragment capable of specifically binding to a CH3 domain of human IgG conjugated to a cross-linked poly(styrene-divinylbenzene) matrix with an alkaline detergent comprising an alkaline salt, one or more surfactants and, optionally, a chelator; and/or
(ii) regenerating a chromatography medium comprising a VHH antibody fragment capable of specifically binding to a CH3 domain of human IgG conjugated to a cross-linked poly(styrene-divinylbenzene) matrix by contacting the medium with a solution comprising a combination of sodium hydroxide and ethanol and sanitizing the medium by contacting the medium with an alkaline detergent comprising an alkaline salt, one or more surfactants and, optionally, a chelator.

22. (canceled)

23. (canceled)

24. The method of claim 1, wherein the method is performed as part of a process of continuous chromatography.

25. A method of regenerating and sanitizing a chromatography medium, the method comprising:
(i) regenerating the chromatography medium by contacting the medium with a regeneration solution comprising ethylene glycol, sodium chloride, sodium hydroxide or a combination of sodium hydroxide and ethanol; and
(ii) sanitizing the chromatography medium by contacting the medium with an alkaline detergent.

26. The method of claim 25, wherein the method comprises:
(i) regenerating the chromatography medium by contacting the medium with a regeneration solution comprising a combination of 5-30 mM sodium hydroxide and 5-30% ethanol; and
(ii) sanitizing the chromatography medium by contacting the medium with an alkaline detergent comprising between 0.01% to 1% (w/v) of one or more surfactants and/or between 60 mM and 80 mM of an alkaline salt selected from sodium hydroxide and potassium hydroxide.

27. The method of claim 25, wherein the alkaline detergent comprises:
(i) between 75 mM of potassium hydroxide or sodium hydroxide and between 0.02% (w/v) of TDAO; and/or
(ii) 2% CIP-100.

28. (canceled)

29. The method of claim 25, wherein the regeneration solution comprises a combination of 5 mM or 20 mM sodium hydroxide and 20% ethanol.
